(19) United States
(12) Patent Application Publication
(10) Pub. No.: US 2019/0016745 A1
(43) Pub. Date: Jan. 17, 2019

(54) AMINOGLYCOSIDE DERIVATIVES AND USES THEREOF IN TREATING MICROBIAL INFECTIONS

(71) Applicant: TECHNION RESEARCH & DEVELOPMENT FOUNDATION LIMITED, Haifa (IL)

(72) Inventor: Timor BAASOV, Haifa (IL)

(73) Assignee: Technion Research & Development Foundation Limited, Haifa (IL)

(21) Appl. No.: 16/068,159

(22) PCT Filed: Sep. 2, 2016
(86) PCT No.: PCT/IL2016/050967

§ 371 Date: Jul. 5, 2018

Related U.S. Application Data
(60) Provisional application No. 62/274,905, filed on Jan. 5, 2016.

(51) Int. Cl. C07H 15/23; A61P 31/04

(52) U.S. Cl. CPC C07H 15/23; A61P 31/04

(57) ABSTRACT

Novel aminoglycosides, represented by Formulae Ia and Ib, as defined in the instant specification, are disclosed. Also disclosed are pharmaceutical compositions containing the same, and uses thereof in the treatment of medical conditions associated with a pathogenic microorganism.

### FIELD AND BACKGROUND OF THE INVENTION

The present invention, in some embodiments thereof, relates to aminoglycosides and more particularly, but not exclusively, to novel aminoglycoside derivatives and to uses thereof in, for example, treatment of medical conditions associated with a pathogenic microorganism.

The rapid spread of antibiotic resistance in pathogenic bacteria has prompted a continuing search for new agents capable of antibacterial activity. Thus, research into the design of new antibiotics is of high priority.

One example of an important group of antibiotics which could benefit from such a design is the aminoglycoside class of antibiotics. Aminoglycosides are highly potent, broad-spectrum antibiotics with many desirable properties for the treatment of life-threatening infections.

Unfortunately, prolonged clinical use of currently available aminoglycosides has resulted in effective selection of resistance to this family of antibacterial agents. Presently, resistance to these agents is widespread among pathogens worldwide which severely limits their usefulness.

It is accepted that the mechanism of action of aminoglycoside antibiotics, such as paromomycin, involves interaction with the prokaryotic ribosome, and, more specifically, involves binding to the decoding A-site of the 16S ribosomal RNA, which leads to protein translation inhibition and interference with the translational fidelity.

Several achievements in bacterial ribosome structure determination, along with crystal and NMR structures of bacterial A-site oligonucleotide models, have provided useful information for understanding the decoding mechanism in prokaryote cells and understanding how aminoglycosides exert their deleterious misreading of the genetic code. These studies and others have given rise to the hypothesis that the affinity of the A-site for a non-cognate mRNA-tRNA complex is increased upon aminoglycoside binding, preventing the ribosome from efficiently discriminating between non-cognate and cognate complexes.

The enhancement of termination suppression by aminoglycosides in eukaryotes is thought to occur in a similar mechanism to the aminoglycosides' activity in prokaryotes of interfering with translational fidelity during protein synthesis, namely the binding of certain aminoglycosides to the ribosomal A-site probably induce conformational changes that stabilize near-cognate mRNA-tRNA complexes, instead of inserting the release factor. Aminoglycosides have been shown to suppress various stop codons with notably different efficiencies (UGA>UAG>UAA), and the suppression effectiveness has been found to be further dependent upon the identity of the fourth nucleotide immediately downstream from the stop codon (C>U>A≥grams) as well as the local sequence context around the stop codon.

To tackle the problem of antibiotic resistance, many structural analogs of natural aminoglycosides have been synthesized over the past decades. In the majority of these studies a minimal structural motif, which is common for a series of structurally related aminoglycosides, has been identified and used as a scaffold for the construction of diverse analogs as potential new antibiotics. Some of the designed structures showed considerable antibacterial activities.

An additional major limitation in using aminoglycosides as pharmaceuticals is their high toxicity towards mammals, typically expressed in kidney (nephrotoxicity) and ear-associated (ototoxicity) illnesses. The origin of this toxicity is assumed to result from a combination of different factors and mechanisms such as interactions with phospholipids, inhibition of phospholipases and the formation of free radicals. Although considered selective to bacterial ribosomes, most aminoglycosides bind also to the eukaryotic A-site but with lower affinities than to the bacterial A-site. The inhibition of translation in mammalian cells is also one of the possible causes for the high toxicity of these agents. Another factor adding to their cytotoxicity is their binding to the mitochondrial ribosome at the 12S rRNA A-site, whose sequence is very close to the bacterial A-site.

Many studies have been attempted to understand and offer ways to alleviate the toxicity associated with aminoglycosides, including the use of antioxidants to reduce free radical levels, as well as the use of poly-L-aspartate and daptomycin, to reduce the ability of aminoglycosides to interact with phospholipids. The role of megalin (a multiligand endocytic receptor which is especially abundant in the kidney proximal tubules and the inner ear) in the uptake of aminoglycosides has recently been demonstrated. The administration of agonists that compete for aminoglycoside binding to megalin also resulted in a reduction in aminoglycoside uptake and toxicity. In addition, altering the administration schedule and/or the manner in which aminoglycosides are administered has been investigated as means to reduce toxicity.

WO 2007/113841 and WO 2012/066546 disclose classes of paromomycin-derived aminoglycosides, designed to exhibit high premature stop codon mutations readthrough activity while exerting low cytotoxicity in mammalian cells and low antimicrobial activity, and can thus be used in the treatment of genetic diseases. This class of paromomycin-derived aminoglycosides was designed by introducing certain manipulations to the paromamine core, which lead to enhanced readthrough activity and reduced toxicity and antimicrobial activity. The manipulations were made on several positions of the paromamine core.

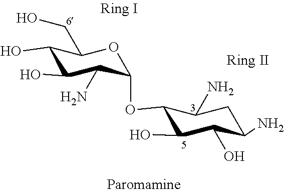

Exemplary such manipulations of the paromamine core which have been taught in these publications include a hydroxyl group at position 6′ of the aminoglycoside core; introduction of one or more monosaccharide moieties or an oligosaccharide moiety at position 3′, 5 and/or 6 of the aminoglycoside core; introduction of an (S)-4-amino-2-hydroxybutyryl (AHB) moiety at position 1 of the paromamine core; substitution of hydrogen at position 6′ by an alkyl such as a methyl substituent; and an introductions of an alkyl group at the 5″ position.

U.S. Pat. No. 7,635,586 discloses aminoglycosides derived from Neomycin B, and their use as highly potent and effective antibiotics, while reducing or even blocking antibiotic resistance.

Additional background art includes Nudelman, I., et al., Bioorg Med Chem Lett, 2006. 16(24): p. 6310-5; Hobbie, S. N., et al., Nucleic Acids Res, 2007. 35(18): p. 6086-93; Kondo, J., et al., Chembiochem, 2007. 8(14): p. 1700-9; Rebibo-Sabbah, A., et al., Hum Genet, 2007. 122(3-4): p. 373-81; Azimov, R., et al., Am J Physiol Renal Physiol, 2008. 295(3): p. F633-41; Hainrichson, M., et al., Org Biomol Chem, 2008.6(2): p. 227-39; Hobbie, S. N., et al., Proc Natl Acad Sci U S A, 2008. 105(52): p. 20888-93; Hobbie, S. N., et al., Proc Natl Acad Sci U S A, 2008. 105(9): p. 3244-9; Nudelman, I., et al., Adv. Synth. Catal., 2008. 350: p. 1682-1688; Nudelman, I., et al., J Med Chem, 2009. 52(9): p. 2836-45; Venkataraman, N., et al., PLoS Biol, 2009. 7(4): p. e95; Brendel, C., et al., J Mol Med (Berl), 2010.89(4): p. 389-98; Goldmann, T., et al., Invest Ophthalmol Vis Sci, 2010. 51(12): p. 6671-80; Malik, V., et al., Ther Adv Neurol Disord, 2010. 3(6): p. 379-89; Nudelman, I., et al., Bioorg Med Chem, 2010. 18(11): p. 3735-46; Warchol, M. E., Curr Opin Otolaryngol Head Neck Surg, 2010. 18(5): p. 454-8; Lopez-Novoa, J. M., et al., Kidney Int, 2011. 79(1): p. 33-45; Rowe, S. M., et al., J Mol Med (Berl), 2011. 89(11): p. 1149-61; Vecsler, M., et al., PLoS One, 2011. 6(6): p. e20733; U.S. Pat. Nos. 3,897,412, 4,024,332, 4,029,882, and 3,996,205; Greenberg et al., J. Am. Chem. Soc., 1999, 121, 6527-6541; Kotra et al., antimicrobial agents and chemotherapy, 2000, p. 3249-3256; Haddad et al., J. Am. Chem. Soc., 2002, 124, 3229-3237; Kandasamy, J. et al., J. Med. Chem. 2012, 55, pp. 10630-10643; Duscha, S. et al., MBio, 2014, 5(5), p. e01827-14; Huth, M. E. et al., J Clin Invest., 2015, 125(2), pp. 583-92; Shulman, E. et al., J Biol Chem., 2014, 289(4), pp. 2318-30 and FR Patent No. 2,427,341, JP Patent No. 04046189. The teachings of all of these documents are incorporated by reference as if fully set forth herein.

### SUMMARY OF THE INVENTION

The present invention relates to aminoglycosides, which can be beneficially used in the treatment of medical conditions associated with pathogenic microorganisms, which are also referred to herein as “microbial infections”. The presently disclosed aminoglycosides are characterized by a core structure based on Rings I, II and optionally III of paromomycin.

According to an aspect of some embodiments of the present invention there is provided a compound represented by Formula I:

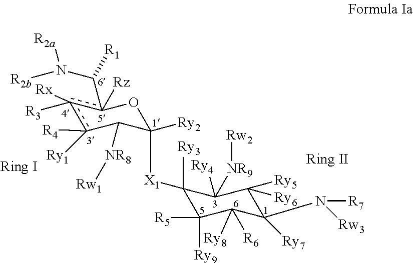

or a pharmaceutically acceptable salt thereof,

wherein:

the dashed line indicates a stereo-configuration of position 6′ being an R configuration or an S configuration;

X₁ is O or S;

the dashed bond between C4′ and C5′ in Ring I represents a single bond or a double bond;

the dashed bond between C4′ and C3′ in Ring I represents a single bond or a double bond;

Rx, Ry1 and Rz are each independently hydrogen, alkyl or cycloalkyl, or absent, wherein Rx and Rz are both absent in case the dashed bond between C4′ and C5′ is a double bond, and Rx and Ry1 are both absent in case the dashed bond between C4′ and C3′ is a double bond;

Ry2-Ry9 and Rw1-Rw3 are each independently selected from hydrogen, alkyl, alkenyl, alkynyl, aryl, heteroaryl and cycloalkyl, each being substituted or unsubstituted, or, alternatively, each can be as defined herein for R₇-R₉;

R₁ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated and/or substituted or unsubstituted hydroxy alkyl (e.g., —CH₂—OH);

R₂ₐ and R_{2b} are each independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted heteroalicyclic and acyl;

R₃-R₆ are each independently selected from the group consisting of hydrogen, alkyl, alkenyl, alkynyl, cycloalkyl, heteroalicyclic, aryl, heteroaryl, amine and OR₁₆, wherein R₁₆ is independently selected from hydrogen, a monosaccharide moiety, an oligosaccharide moiety, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl and acyl; and

R₇-R₉ are each independently selected from the group consisting of hydrogen, acyl, an amino-substituted alpha-hydroxy acyl, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted alkaryl, carboxylate, sulfonyl (including alkyl sulfonyl and aryl sulfonyl) and a cell-permealizable group.

According to some of any of the embodiments described herein, at least one of R₃-R₆ is OR₁₆.

According to some of any of the embodiments described herein, R₁₆ an aryl.

According to some of any of the embodiments described herein, at least one of R₃-R₆ is selected from the group consisting of phenyloxy, 1-anthryloxy, 1-naphthyloxy, 2-naphthyloxy, 2-phenanthryloxy and 9-phenanthryloxy.

According to some of any of the embodiments described herein, R₁₆ is a substituted or unsubstituted heteroaryl, and at least one of R₃-R₆ is independently a substituted or unsubstituted heteroaryloxy.

According to some of any of the embodiments described herein, at least one of R₃-R₆ is independently selected from the group consisting of 2-anthryloxy, 2-furyloxy, 2-indolyloxy, 2-naphthyloxy, 2-pyridyloxy, 2-pyrimidyloxy, 2-pyrryloxy, 2-quinolyloxy, 2-thienyloxy, 3-furyloxy, 3-indolyloxy, 3-thienyloxy, 4-imidazolyloxy, 4-pyridyloxy, 4-pyrimidyloxy, 4-quinolyloxy, 5-methyl-2-thienyloxy and 6-chloro-3-pyridyloxy.

According to some of any of the embodiments described herein, R₁₆ is a substituted aryl.

According to some of any of the embodiments described herein, at least one of R₃-R₆ is OR₁₆, and R₁₆ is independently selected from the group consisting of 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and 4-valeroyloxycarbonylphenyl.

According to some of any of the embodiments described herein, R₃ is OR₁₆ and R₁₆ is hydrogen.

According to some of any of the embodiments described herein, R₃ is OR₁₆ and R₁₆ is selected from the group consisting of methyl, ethyl, propyl, butyl, pentyl, propenyl, 2-hydroxyethyl, 3-hydroxypropyl, 2,3-dihydroxypropyl and methoxymethyl.

According to some of any of the embodiments described herein, R₄ is OR₁₆ and R₁₆ is hydrogen.

According to some of any of the embodiments described herein, each of R₃ and R₄ is OR₁₆ and R₁₆ is hydrogen.

According to some of any of the embodiments described herein, at least one of R₃-R₆ is OR₁₆ and R₁₆ is independently an acyl.

According to some of any of the embodiments described herein, at least one of R₃-R₆ is OR₁₆ in which R₁₆ is a monosaccharide moiety.

According to some of any of the embodiments described herein, the monosaccharide moiety is represented by Formula II:

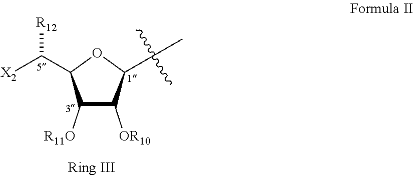

wherein:

the curved line denotes a position of attachment;

the dashed line indicates a stereo-configuration of position 5″ being an R configuration or an S configuration;

X₂ is OR₁₃ or NR₁₄R₁₅;

each of R₁₀, R₁₁ and R₁₃ is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, and acyl;

R₁₂ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated and/or substituted or unsubstituted hydroxyalkyl;

each of R₁₄-and R₁₅ is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, acyl, and a cell-permealizable group, or, alternatively, R₁₄ and R₁₅, when present, form together a heterocyclic ring.

Substituents not shown in Formula II at positions 6′, 1″, 2″, 3″, 4″ and 5″ are typically hydrogen, although other substituents, such as, but not limited, as defined for Ry2-Ry9, are also contemplated.

According to some of any of the embodiments described herein, the compound as described herein is represented by Formula Ib:

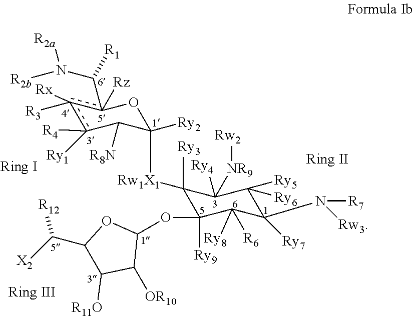

Substituents not shown in Formula Ib at positions 6′ 1″, 2″, 3″, 4″ and 5″ are typically hydrogen, although other substituents, such as, but not limited, as defined for Ry2-Ry9, are also contemplated.

According to some of any of the embodiments described herein, X₂ is OR₁₃.

According to some of any of the embodiments described herein, X₂ is NR₁₄R₁₅.

According to some of any of the embodiments described herein, R₁₂ is other than hydrogen.

According to some of any of the embodiments described herein, at least one of R₁₀, R₁₁ and R₁₃, if present, is an acyl.

According to some of any of the embodiments described herein, X₁ is O.

According to some of any of the embodiments described herein, the bond between C4′ and C5′ in Ring I is a single bond.

According to some of any of the embodiments described herein, the bond between C4′ and C5′ in Ring I is a double bond and Rx and Rz are absent.

According to some of any of the embodiments described herein, the bond between C4′ and C3′ in Ring I is a single bond.

According to some of any of the embodiments described herein, the bond between C4′ and C3′ in Ring I is a double bond and Rx and Ry1 is absent.

According to some of any of the embodiments described herein, R₁ is other than hydrogen.

According to some of any of the embodiments described herein, R₁ is a hydroxyalkyl.

According to some of any of the embodiments described herein, R₁ is a substituted or unsubstituted alkyl, a substituted or unsubstituted alkenyl or a substituted or unsubstituted alkynyl.

According to some of any of the embodiments described herein, R₁ is selected from the group consisting of methyl, ethyl, propyl, butyl and pentyl.

According to some of any of the embodiments described herein, R₁ is or comprises an aryl.

According to some of any of the embodiments described herein, R₁ is selected from the group consisting of phenyl, 1-anthryl, 1-naphthyl, 2-naphthyl, 2-phenanthryl and 9-phenanthryl.

According to some of any of the embodiments described herein, R₁ is or comprises a substituted or unsubstituted heteroaryl.

According to some of any of the embodiments described herein, R₁ is selected from the group consisting of 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and 6-chloro-3-pyridyl.

According to some of any of the embodiments described herein, R₁ is a substituted aryl.

According to some of any of the embodiments described herein, R₁ is selected from the group consisting of 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and 4-valeroyloxycarbonylphenyl.

According to some of any of the embodiments described herein, R₁ is amine.

According to some of any of the embodiments described herein, R₁ is selected from the group consisting of —NH₂, —NHCH₃, —N(CH₃)₂, —NH—CH₂—CH₂—NH₂, —NH—CH₂—CH₂—OH and —NH—CH₂—CH(OCH₃)₂.

According to some of any of the embodiments described herein, each of R₂ₐ and R_{2b} is hydrogen.

According to some of any of the embodiments described herein, one or both of R₂ₐ and R_{2b} is alkyl, preferably selected from the group consisting of methyl, ethyl and propyl.

According to some of any of the embodiments described herein, at least one of R₂ₐ and R_{2b} is a substituted or unsubstituted alkyl, which can be represented by:

—(CH₂)n—NH₂;

—(CH₂)n—C(═O)R′″;

—(CH₂)n—CH(OR′″)₂; or

—(CH₂)n-R″

wherein n is a positive integer, preferably of from 1 to 6;

and R′″ is hydrogen, alkyl, cycloalkyl, aryl, heteroaryl or heteroalicyclic.

According to some of any of the embodiments described herein, R₇ is selected from the group consisting of hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, -β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(C H₂)₂NH-ethyl, —(CH₂)₂NH(CH₂)₂NH₂, —(CH₂)₃NH(CH₂)₃NH₂, —(CH₂)₃NH(CH₂)₄NH(CH ₂)₃NH₂, —CH(—NH₂)CH₂(OH), —CH(—OH)CH₂(NH₂), —CH(—OH)—(CH₂)₂(NH₂), —CH(—NH₂) —(CH₂)₂(OH), —CH(—CH₂NH₂)—(CH₂OH), —(CH₂)₄NH(CH₂)₃NH₂, —(CH₂)₂NH(CH₂)₂NH( CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)—C(═O)NH₂, —CH(benzyl)-C(═O)NH₂, —(CH₂)₂OH, —(CH₂)₃OH and —CH(CH₂OH)₂.

According to some of any of the embodiments described herein, each of R₈ and R₉ is independently selected from the group consisting of hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —COO-methyl, —COO-ethyl, —COO-benzyl, -β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(C H₂)₂NH-ethyl, —(CH₂)₂NH(CH₂)₂NH₂, —(CH₂)₃NH(CH₂)₃NH₂, —(CH₂)₃NH(CH₂)₄NH(CH ₂)₃NH₂, —CH(—NH₂)CH₂(OH), —CH(—OH)CH₂(NH₂), —CH(—OH)—(CH₂)₂(NH₂), —CH(—NH₂) —(CH₂)₂(OH), —CH(—CH₂NH₂)—(CH₂OH), —(CH₂)₄NH(CH₂)₃NH₂, —(CH₂)₂NH(CH₂)₂NH( CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

According to some of any of the embodiments described herein, the amino-substituted alpha-hydroxy acyl is (S)-4-amino-2-hydroxybutyryl (AHB).

According to some of any of the embodiments described herein, the cell-permealizable group is guanidyl.

According to some of any of the embodiments described herein, the unsubstituted aryl is selected from the group consisting of phenyl, 1-anthryl, 1-naphthyl, 2-naphthyl, 2-phenanthryl and 9-phenanthryl.

According to some of any of the embodiments described herein, the substituted or unsubstituted heteroaryl is selected from the group consisting of 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and 6-chloro-3-pyridyl.

According to some of any of the embodiments described herein, the substituted aryl is selected from the group consisting of 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and 4-valeroyloxycarbonylphenyl.

According to some of any of the embodiments described herein, the acyl is selected from the group consisting of a hydrocarbon acyl radical having from 2 to 18 carbon atoms, optionally substituted by one or more of halo, nitro, hydroxy, amine, cyano, thiocyano, and alkoxy.

According to some of any of the embodiments described herein, the acyl is derived from an acid selected from the group consisting of a saturated or unsaturated and/or substituted or unsubstituted aliphatic carboxylic acid, acetic acid, propionic acid, butyric acid, isobutyric acid, tert-butylacetic acid, valeric acid, isovaleric acid, caproic acid, caprylic acid, decanoic acid, dodecanoic acid, lauric acid, tridecanoic acid, myristic acid, pentadecanoic acid, palmitic acid, margaric acid, stearic acid, acrylic acid, crotonic acid, undecylenic acid, oleic acid, hexynoic acid, heptynoic acid, octynoic acid, a saturated or unsaturated alicyclic carboxylic acid, cyclobutanecarboxylic acid, cyclopentanecarboxylic acid, cyclopentenecarboxylic acid, methylcyclopentenecarboxylic acid, cyclohexanecarboxylic acid, dimethylcyclohexanecarboxylic acid, dipropylcyclohexanecarboxylic acid, a saturated or unsaturated, alicyclic aliphatic carboxylic acid, cyclopentaneacetic acid, cyclopentanepropionic acid, cyclohexaneacetic acid, cyclohexanebutyric acid, methylcyclohexaneacetic acid, a substituted or unsubstituted aromatic carboxylic acid, benzoic acid, toluic acid, naphthoic acid, ethylbenzoic acid, isobutylbenzoic acid, methylbutylbenzoic acid, an aromatic aliphatic carboxylic acid, phenylacetic acid, phenylpropionic acid, phenylvaleric acid, cinnamic acid, phenylpropiolic acid, naphthylacetic acid, a halo-alkoxyhydrocarbon carboxylic acid, a nitro-alkoxyhydrocarbon carboxylic acid, a hydroxy-alkoxyhydrocarbon carboxylic acid, an amino-alkoxyhydrocarbon carboxylic acid, a cyano-alkoxyhydrocarbon carboxylic acid, a thiocyano-alkoxyhydrocarbon carboxylic acid, mono-acetic acid, di-acetic acid, trichloroacetic acid, 1,2,3,4,5,6-hexachlorocyclohexanecarboxylic acid, 1,2-dibromo-4-methylcyclohexanecarboxylic acid, 1,6-dibromo-3-methylcyclohexanecarboxylic acid, 1-bromo-3,5-dimethylcyclohexanecarboxylic acid, 2--chlorocyclohexanecarboxylic acid, 4-chlorocyclohexanecarboxylic acid, 2,3-dibromo-2-methylcyclohexanecarboxylic acid, 2,4,6-trinitrobenzoic acid, 2,5-dibromo-2-methylcyclohexanecarboxylic acid, 2-bromo-4-methylcyclohexanecarboxylic acid, 2-nitro-1-methyl-cyclobutanecarboxylic acid, 3,4-dinitrobenzoic acid, 3,5-dinitrobenzoic acid, 3-bromo-2,2,3-trimethylcyclopentanecarboxylic acid, 3-bromo-2-methylcyclohexanecarboxylic acid, 3-bromo-3-methylcyclohexanecarboxylic acid, 4-bromo-2-methylcyclohexanecarboxylic acid, 5-bromo-2-methylcyclohexanecarboxylic acid, ‘4,4-dichlorobenzilic acid, 4,5-dibromo-2-methylcyclohexanecarboxylic acid, 5-bromo-2-methylcyclohexanecarboxylic acid, 6-bromo-2-methylcyclohexanecarboxylic acid, 5,6-dibromo-2-methylcyclohexanecarboxylic acid, 6-bromo-3-methylcyclohexanecarboxylic acid, anisic acid, cyanoacetic acid, cyanopropionic acid, ethoxyformic acid (ethyl hydrogen carbonate), gallic acid, homogentisic acid, o-, m-, and p-chlorobenzoic acid, lactic acid, mevalonic acid, o-, m-, p-nitrobenzoic acid, p-hydroxybenzoic acid, salicyclic acid, shikimic acid, thiocyanoacetic acid, trimethoxybenzoic acid, trimethoxycinnamic acid, veratric acid, α- and β-chloropropionic acid, α- and γ-bromobutyric acid and α- and δ-iodovaleric acid, β-resorcylic acid.

According to an aspect of some embodiments of the present invention, there is provided a pharmaceutical composition comprising the compound as described in any one of the embodiments described herein and any combination thereof and a pharmaceutically acceptable carrier.

According to some of any of the embodiments described herein, the pharmaceutical composition is packaged in a packaging material and identified in print, in or on the packaging material, for use in the treatment of medical condition associated with a pathogenic microorganism.

According to an aspect of some embodiments of the present invention there is provided a method for treating a medical condition associated with a pathogenic microorganism, the method comprising administering to a subject in need thereof a therapeutically effective amount of the compound as described in any one of the embodiments described herein and any combination thereof.

According to an aspect of some embodiments of the present invention, there is provided a compound as described in any one of the embodiments described herein and any combination thereof, for use in the treatment of a medical condition associated with a pathogenic microorganism.

According to an aspect of some embodiments of the present invention, there is provided a use of the compound as described in any one of the embodiments described herein and any combination thereof in the manufacture of a medicament for treating medical condition associated with a pathogenic microorganism.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to aminoglycosides, and, more particularly, but not exclusively, to novel aminoglycoside derivatives and to uses thereof in, for example, treatment of medical conditions associated with a pathogenic microorganism.

Specifically, the present invention, in some embodiments thereof, relates to a novel aminoglycoside compounds, derived from paromomycin. Embodiments of the present invention are further of pharmaceutical compositions containing these compounds, and of uses thereof in the treatment of medical conditions associated with pathogenic microorganisms, also referred to herein as “microbial infections”.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The Compounds:

According to an aspect of some embodiments of the present invention, there are provided novel aminoglycoside compounds (also referred to herein as “aminoglycoside derivatives”, which are collectively represented by Formula Ia:

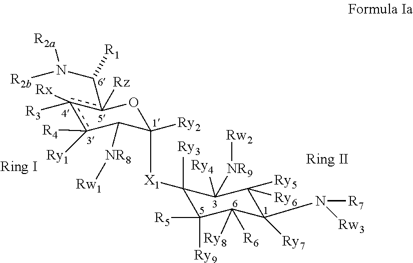

wherein:

the dashed line indicates a stereo-configuration of position 6’ being an R configuration or an S configuration;

X₁ is O or S;

the dashed bond between C4′ and C5′ in Ring I represents a single bond or a double bond;

the dashed bond between C4′ and C3′ in Ring I represents a single bond or a double bond;

Rx, Ry1 and Rz are each independently selected from hydrogen, alkyl, alkenyl, alkynyl, alkaryl, aryl heteroaryl and cycloalkyl, or absent, wherein Rx and Rz are both absent in case the dashed bond between C4′ and C5′ is a double bond, and Rx and Ry1 are both absent in case the dashed bond between C4′ and C3′ is a double bond;

Ry2-Ry9 and Rw1-Rw3 are each independently selected from hydrogen, alkyl, alkenyl, alkynyl, alkaryl, aryl, heteroaryl and cycloalkyl, each being substituted or unsubstituted, or, alternatively, each can be as defined herein for R₇-R₉;

R₁ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated, a substituted or unsubstituted hydroxy alkyl (e.g., —CH₂—OH) and a substituted or unsubstituted aminoalkyl;

R₂ₐ and R_{2b} are each independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroalicyclic; a substituted or unsubstituted heteroaryl, a substituted or unsubstituted alkaryl and acyl;

R₃-R₆ are each independently selected from the group consisting of hydrogen, alkyl, alkenyl, alkynyl, cycloalkyl, heteroalicyclic, aryl, heteroaryl, amine and OR₁₆, wherein R₁₆ is independently (when 2 or more of R₃-R₆ is said OR₁₆) selected from a monosaccharide moiety, an oligosaccharide moiety, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl and acyl; and

R₇-R₉ are each independently selected from the group consisting of hydrogen, acyl, an amino-substituted alpha-hydroxy acyl, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted alkaryl, carboxylate, sulfonyl (including alkyl sulfonyl and aryl sulfonyl) and a cell-permealizable group.

In some of any of the embodiments described herein, the compound is a pseudo-disaccharide, having Ring I and Ring II as depicted in Formula Ia.

In these embodiments, none of R₃-R₆ is OR₁₆ in which R₁₆ is a monosaccharide or an oligosaccharide moiety.

In some of these embodiments, one or more, or all of R₃-R₆ is OR₁₆.

In some of these embodiments, one or more, or all of R₃-R₆ is OR₁₆, and R₁₆ is hydrogen.

In some of these embodiments, R₃ is OR₁₆, and R₁₆ is hydrogen.

In some of these embodiments, one or more, or all of R₃-R₆ is OR₁₆, and in one or more, or all, of R₃-R₆, R₁₆ is independently alkyl or aryl.

In some of these embodiments, one or more, or all of R₃-R₆ is OR₁₆ and R₁₆ is independently an aryl, which can be substituted or unsubstituted. In these embodiments, one or more, or all of R₃-R₆ is an aryloxy, as defined herein.

In some of these embodiments, the aryl is unsubstituted such that one or more, or all of R₃-R₆, independently, can be, as non-limiting examples, phenyloxy, 1-anthryloxy, 1-naphthyloxy, 2-naphthyloxy, 2-phenanthryloxy and 9-phenanthryloxy.

In some of these embodiments, one or more of the aryls in one or more of OR₁₆ is a substituted aryl, such that one or more, or all of R₃-R₆, independently, can be, as non-limiting examples, an aryloxy in which the aryl is 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

In some of these embodiments, one or more, or all of R₃-R₆ is OR₁₆ and R₁₆ is independently a heteroaryl, which can be substituted or unsubstituted. In these embodiments, one or more, or all of R₃-R₆ is a heteroaryloxy, as defined herein.

In some embodiments, one or more, or all of R₃-R₆, independently, can be, as non-limiting examples, 2-anthryloxy, 2-furyloxy, 2-indolyloxy, 2-naphthyloxy, 2-pyridyloxy, 2-pyrimidyloxy, 2-pyrryloxy, 2-quinolyloxy, 2-thienyloxy, 3-furyloxy, 3-indolyloxy, 3-thienyloxy, 4-imidazolyloxy, 4-pyridyloxy, 4-pyrimidyloxy, 4-quinolyloxy, 5-methyl-2-thienyloxy and 6-chloro-3-pyridyloxy.

In some of any of the embodiments described herein, R₃ is aryloxy or heteroaryloxy, as described herein.

In some of any of the embodiments described herein, R₃ is OR₁₆ and R₁₆ is a substituted or unsubstituted alkyl or alkenyl, for example, methyl, ethyl, propyl, butyl, pentyl, propenyl, 2-hydroxyethyl, 3-hydroxypropyl, 2,3-dihydroxypropyl and methoxymethyl.

In some of any of the embodiments described herein, R₃ is OR₁₆ and R₁₆ is hydrogen.

In some of any of the embodiments described herein, one or more of, or all of R₃-R₆ are OR₁₆.

In some of these embodiments, in each of R₃-R₆, R₁₆ is hydrogen.

In some of these embodiments, in one or more, or all, of R₃-R₆, R₁₆ is other than hydrogen.

In some of any of the embodiments described herein, when one or more, or all, of R₃-R₆ is OR₁₆ and when one or more, or all of the R₁₆ moiety is other than hydrogen, R₁₆ can be the same or different for each of R₃-R₆.

In some of these embodiments, when in one or more, or all, of R₃-R₆, R₁₆ is other than hydrogen, R₁₆ can be, for example, independently, alkyl, alkenyl, alkynyl, cycloalkyl, aryl or heteroaryl, each being optionally substituted, as described herein.

In some of any of the embodiments described herein, in one or more, or all, of R₃-R₆, R₁₆ is independently an acyl, forming an ester (a carboxylate) at the respective position.

Herein throughout, the term “acyl” describes a —C(═O)—R group, wherein R is as described herein.

Herein throughout, the term “acyl” describes a —C(═O)—R group, with R being a substituted or unsubstituted alkyl, cycloalkyl, aryl, alkaryl, a hydrocarbon chain, or hydrogen.

In exemplary embodiments, the acyl is such that R is an alkyl or alkaryl or aryl, each of which being optionally substituted by one or more amine substituents.

In some embodiments, R is a substituted alkyl, and in some embodiments, R is substituted by hydroxy at the α position with respect to the carbonyl group, such that the acyl is α-hydroxy-acyl.

In some embodiments, the α-hydroxy-acyl is further substituted by one or more amine groups, and is an amino-substituted α-hydroxy-acyl.

In some of the embodiments of an acyl group as described herein, the amine substituents can be, for example, at one or more of positions β, γ, δ, and/or ω of the moiety R, with respect to the acyl.

Exemplary amino-substituted α-hydroxy-acyls include, without limitation, the moiety (S)-4-amino-2-hydroxybutyryl, which is also referred to herein as AHB. According to some embodiments of the present invention, an alternative to the AHB moiety can be the α-hydroxy-β-aminopropionyl (AHP) moiety. Additional exemplary amino-substituted α-hydroxy-acyls include, but are not limited to, L-(-)-γ-amino-α-hydroxybutyryl, L(-)-δ-amino-α-hydroxyvaleryl, L-(-)-β-benzyloxycarbonylamino-α-hydroxypropionyl, a L-(-)-δ-benzyloxycarbonylamino-α-hydroxyvaleryl.

It is noted herein that according to some embodiments of the present invention, other moieties which involve a combination of carbonyl(s), hydroxyl(s) and amino group(s) along a lower alkyl exhibiting any stereochemistry, are contemplated as optional substituents in place of AHB and/or AHP, including, for example, 2-amino-3-hydroxybutanoyl, 3-amino-2-hydroxypentanoyl, 5-amino-3-hydroxyhexanoyl and the likes.

In some of any of the embodiments described herein, R is a hydrocarbon chain, as described herein, optionally substituted. In some embodiments, the hydrocarbon chain is of 2 to 18 carbon atoms in length. In some embodiments, the acyl is a hydrocarbon acyl radical having from 2 to 18 carbon atoms, optionally substituted by one or more of halo, nitro, hydroxy, amine, cyano, thiocyano, and alkoxy.

Herein, the term “hydrocarbon” describes an organic moiety that includes, as its basic skeleton, a chain of carbon atoms, also referred to herein as a backbone chain, substituted mainly by hydrogen atoms. The hydrocarbon can be saturated or non-saturated, be comprised of aliphatic, alicyclic and/or aromatic moieties, and can optionally be substituted by one or more substituents (other than hydrogen). A substituted hydrocarbon may have one or more substituents, whereby each substituent group can independently be, for example, alkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, azide, sulfonamide, carboxy, thiocarbamate, urea, thiourea, carbamate, amide, and hydrazine, and any other substituents as described herein.

The hydrocarbon moiety can optionally be interrupted by one or more heteroatoms, including, without limitation, one or more oxygen, nitrogen (substituted or unsubstituted, as defined herein for —NR′—) and/or sulfur atoms.

In some embodiments of any of the embodiments described herein relating to a hydrocarbon, the hydrocarbon is not interrupted by any heteroatom, nor does it comprise heteroatoms in its backbone chain, and can be an alkylene chain, or be comprised of alkyls, cycloalkyls, aryls, alkenes and/or alkynes, covalently attached to one another in any order.

In some of any of the embodiments described herein, the acyl is derived from a carboxylic acid, such that the ester formed at the respective position is derived from, for example a saturated or unsaturated and/or substituted or unsubstituted aliphatic carboxylic acid, acetic acid, propionic acid, butyric acid, isobutyric acid, tert-butylacetic acid, valeric acid, isovaleric acid, caproic acid, caprylic acid, decanoic acid, dodecanoic acid, lauric acid, tridecanoic acid, myristic acid, pentadecanoic acid, palmitic acid, margaric acid, stearic acid, acrylic acid, crotonic acid, undecylenic acid, oleic acid, hexynoic acid, heptynoic acid, octynoic acid, a saturated or unsaturated alicyclic carboxylic acid, cyclobutanecarboxylic acid, cyclopentanecarboxylic acid, cyclopentenecarboxylic acid, methylcyclopentenecarboxylic acid, cyclohexanecarboxylic acid, dimethylcyclohexanecarboxylic acid, dipropylcyclohexanecarboxylic acid, a saturated or unsaturated, alicyclic aliphatic carboxylic acid, cyclopentaneacetic acid, cyclopentanepropionic acid, cyclohexaneacetic acid, cyclohexanebutyric acid, methylcyclohexaneacetic acid, a substituted or unsubstituted aromatic carboxylic acid, benzoic acid, toluic acid, naphthoic acid, ethylbenzoic acid, isobutylbenzoic acid, methylbutylbenzoic acid, an aromatic aliphatic carboxylic acid, phenylacetic acid, phenylpropionic acid, phenylvaleric acid, cinnamic acid, phenylpropiolic acid, naphthylacetic acid, a halo-alkoxyhydrocarbon carboxylic acid, a nitro-alkoxyhydrocarbon carboxylic acid, a hydroxy-alkoxyhydrocarbon carboxylic acid, an amino-alkoxyhydrocarbon carboxylic acid, a cyano-alkoxyhydrocarbon carboxylic acid, a thiocyano-alkoxyhydrocarbon carboxylic acid, mono-acetic acid, di-acetic acid, trichloroacetic acid, 1,2,3,4,5,6-hexachlorocyclohexanecarboxylic acid, 1,2-dibromo-4-methylcyclohexanecarboxylic acid, 1,6-dibromo-3-methylcyclohexanecarboxylic acid, 1-bromo-3,5-dimethylcyclohexanecarboxylic acid, 2-chlorocyclohexanecarboxylic acid, 4-chlorocyclohexanecarboxylic acid, 2,3-dibromo-2-methylcyclohexanecarboxylic acid, 2,4,6-trinitrobenzoic acid, 2,5-dibromo-2-methylcyclohexanecarboxylic acid, 2-bromo-4-methylcyclohexanecarboxylic acid, 2-nitro-1-methyl-cyclobutanecarboxylic acid, 3,4-dinitrobenzoic acid, 3,5-dinitrobenzoic acid, 3-bromo-2,2,3-trimethylcyclopentanecarboxylic acid, 3-bromo-2-methylcyclohexanecarboxylic acid, 3-bromo-3-methylcyclohexanecarboxylic acid, 4-bromo-2-methylcyclohexanecarboxylic acid, 5-bromo-2-methylcyclohexanecarboxylic acid, ‘4,4-dichlorobenzilic acid, 4,5-dibromo-2-methylcyclohexanecarboxylic acid, 5-bromo-2-methylcyclohexanecarboxylic acid, 6-bromo-2-methylcyclohexanecarboxylic acid, 5,6-dibromo-2-methylcyclohexanecarboxylic acid, 6-bromo-3-methylcyclohexanecarboxylic acid, anisic acid, cyanoacetic acid, cyanopropionic acid, ethoxyformic acid (ethyl hydrogen carbonate), gallic acid, homogentisic acid, o-, m-, and p-chlorobenzoic acid, lactic acid, mevalonic acid, o-, m-, p-nitrobenzoic acid, p-hydroxybenzoic acid, salicyclic acid, shikimic acid, thiocyanoacetic acid, trimethoxybenzoic acid, trimethoxycinnamic acid, veratric acid, α- and β-chloropropionic acid, α- and γ-bromobutyric acid and α- and δ-iodovaleric acid, β-resorcylic acid.

In some of any of the embodiments described herein, one or more of R₃-R₆ is other than OR₁₆. In some of any of the embodiments described herein, one or more of R₃-R₆ is hydrogen.

In some of any of the embodiments described herein, R₃ is hydrogen.

In some of any of the embodiments described herein, R₄ is hydrogen.

In some of any of the embodiments described herein R₃ and R₄ are each hydrogen.

In some of any of the embodiments described herein, one or more of R₃-R₆ is OR₁₆ and R₁₆ is independently a monosaccharide moiety or an oligosaccharide moiety, as defined herein, such that the compound is a pseudo-trisaccharide, a pseudo-tetrasaccharide, a pseudo-pentasaccharide, a pseudo hexasaccharide, etc.

The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides like sucrose (common sugar) and other polysaccharides (such as cellulose and starch).

The term “oligosaccharide” as used herein refers to a compound that comprises two or more monosaccharide units, as these are defined herein, linked to one another via a glycosyl bond (—O—). Preferably, the oligosaccharide comprises 2-6 monosaccharides, more preferably the oligosaccharide comprises 2-4 monosaccharides and most preferably the oligosaccharide is a disaccharide moiety, having two monosaccharide units.

In some of any of the embodiments described herein, the monosaccharide is a pentose moiety, such as, for example, represented by Formula II. Alternatively, the monosaccharide moiety is hexose. Further alternatively, the monosaccharide moiety is other than pentose or hexose, for example, a hexose moiety as described in U.S. Pat. No. 3,897,412.

In some of any of the embodiments described herein, the monosaccharide moiety is a ribose, represented by Formula II:

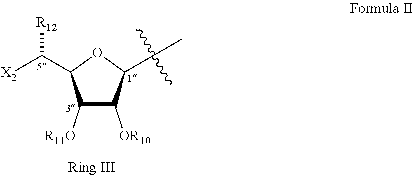

wherein:

the curved line denotes a position of attachment;

the dashed line indicates a stereo-configuration of position 5″ being an R configuration or an S configuration;

X₂ is OR₁₃ or NR₁₄R₁₅;

each of R₁₀, R₁₁ and R_is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, and acyl;

R₁₂ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated and/or substituted or unsubstituted hydroxyalkyl; and

each of R₁₄-and R₁₅ is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, acyl, and a cell-permealizable group, or, alternatively, R₁₄ and R₁₅, when present, form together a heterocyclic ring.

In some embodiments, X₂ is OR₁₃.

In some embodiments, X₂ is NR₁₄R₁₅.

In some of any of the embodiments described herein, R₁₂ is other than hydrogen.

In some of these embodiments, R₁₂ is alkyl, cycloalkyl or aryl, and in some embodiments, R₁₂ is alkyl, preferably a lower alkyl, for example, methyl.

In some embodiments, R₁₂ is as defined herein for R₁.

In some of any of the embodiments where one or more of R₃-R₆ is OR₁₆ and R₁₆ is a monosaccharide moiety or an oligosaccharide moiety, one or more of the hydroxy groups in the monosaccharide or oligosaccharide moiety/moieties are substituted by an acyl, forming an ester (a carboxylate), as described herein in any of the respective embodiments.

In some of any of the embodiments described herein, one of R₃-R₆ is OR₁₆ and R₁₆ is a monosaccharide moiety such that the compound is a pseudo-trisaccharide.

In some of any of the embodiments described herein for a pseudo-trisaccharide, one or more, or all, of R₁₀ and R₁₁, and R₁₃ if present, can be an acyl, as described herein.

In some of any of the embodiments described herein for a pseudo-trisaccharide, one or more, or all, of R₃-R₆ are OR₁₆, such that in one of R₃-R₆, R₁₆ is a monosaccharide moiety, and in the others, R₁₆ is as defined herein (e.g., hydrogen).

In some of any of the embodiments described herein, R₅ is OR₁₆ in which R₁₆ is a monosaccharide moiety.

In some of these embodiments, the compound is represented by Formula Ib:

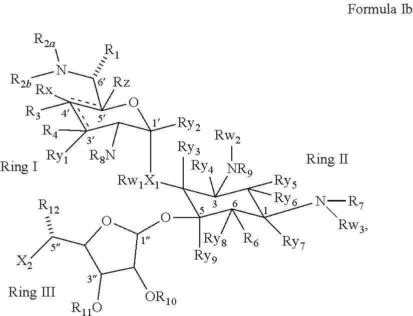

with the variables being as described herein for Formulae Ia and II, including any combination thereof.

In some of any of the embodiments described herein for Formulae Ia and Ib, X₁ is O.

In some of any of the embodiments described herein, the bond between C4′ and C5′ in Ring I is a single bond.

In some of any of the embodiments described herein, the bond between C4′ and C5′ in Ring I is a double bond and Rx and Rz are absent.

In some of any of the embodiments described herein, the bond between C4′ and C3′ in Ring I is a single bond.

In some of any of the embodiments described herein, the bond between C4′ and C3′ in Ring I is a double bond and Rx and Ry1 are absent.

In some of any of the embodiments described herein, one or more, or all, of Rx, Rz, Ry1, if present, and Ry2-Ry9 and Rw1-Rw3 is hydrogen.

In some of any of the embodiments described herein, R₁ is other than hydrogen.

In some of any of the embodiments described herein, R₁ is a hydroxyalkyl, wherein the alkyl can be further substituted or not.

In some of any of the embodiments described herein, R₁ is a hydroxymethyl.

In some of any of the embodiments described herein, R₁ is alkyl, alkenyl or alkynyl, each being substituted or unsubstituted.

In some of any of the embodiments described herein, R₁ is alkyl, preferably a lower alkyl, for example, methyl, ethyl, propyl, butyl or pentyl.

In some of any of the embodiments described herein, R₁ is aryl which can be substituted or unsubstituted. In some embodiments, R₁ is an unsubstituted aryl and can be, as non-limiting examples, phenyl, 1-anthryl, 1-naphthyl, 2-naphthyl, 2-phenanthryl or 9-phenanthryl.

In some embodiments, R₁ is a substituted aryl, and can be, as non-limiting examples, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl or 4-valeroyloxycarbonylphenyl.

In some of any of the embodiments described herein, R₁ is a substituted or unsubstituted heteroaryl, and can be, as non-limiting examples, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and 6-chloro-3-pyridyl.

In some of any of the embodiments described herein, R₁ is amine, as defined herein, and can be, as non-limiting examples, —NH₂, —NHCH₃, —N(CH₃)₂, —NH—CH₂—CH₂—NH₂, —NH—CH₂—CH₂—OH and —NH—CH₂—CH(OCH₃)₂.

In some of any of the embodiments described herein, R₁ is alkyl, and in some embodiments it is a lower alkyl, of 1 to 4 carbon atoms, including, but not limited to, methyl, ethyl, propyl, butyl, isopropyl, and isobutyl.

In some of any of the embodiments described herein, R₁ is a non-substituted alkyl.

In some of any of the embodiments described herein, R₁ is methyl.

Alternatively, in some of any of the embodiments described herein, R₁ is cycloalkyl, including, but not limited to, cyclopropyl, cyclobutyl, cyclopentyl and cyclohexyl.

Further alternatively, in some of any of the embodiments described herein, R₁ is aryl, such as substituted or unsubstituted phenyl. Non-limiting examples include unsubstituted phenyl and toluene.

Further alternatively, in some of any of the embodiments described herein, R₁ is alkaryl, such as substituted or unsubstituted benzyl.

In some of any of the embodiments described herein, R₁ is other than alkyl, cycloalkyl and aryl.

In some of any of the embodiments described herein, R₁ is other than alkyl, cycloalkyl and aryl, wherein each is unsubstituted.

In some of any of the embodiments described herein, R₁ is other than methyl.

In some of any of the embodiments described herein, R₁ is aminoalkyl.

In some of any of the embodiments described herein, one or both of R₂ₐ and R_{2b} is hydrogen.

In some of these embodiments, one or both of R₂ₐ and R_{2b} is other than hydrogen.

Alternatively, one or both of R₂ₐ and R_{2b} is independently an alkyl, for example, methyl, ethyl and/or propyl.

In some of any of the embodiments described herein, one or both of R₂ₐ and R_{2b} is independently an alkyl, and in some of these embodiments one or both of R₂ₐ and R_{2b} is independently a substituted alkyl, for example, an alkyl substituted by one or more amine groups (aminoalkyl).

In some embodiments one or both of R₂ₐ and R_{2b} is independently an alkyl amino, such as —(CH₂)n-NH₂, with n being, for example, from 1 to 6; or a hydroxyalkyl such as —(CH₂)n-OH, with n being, for example, from 1 to 6; or a carboxy alkyl such as —(CH₂)n-C (═O)R′″, with n being, for example, from 1 to 6, and R′″ being hydrogen or alkyl or cycloalkyl or aryl; or an alkoxy alkyl such as —(CH₂)n-CH(OR′″)₂, with n being, for example, from 1 to 6, and R′″ being hydrogen or alkyl or cycloalkyl or aryl; or another substituted or unsubstituted alkyl such as —(CH₂)n-R′″, with n being, for example, from 1 to 6, and R′″ being hydrogen or alkyl or cycloalkyl or aryl or heteroaryl or heteroalicyclic.

In some of these embodiments, one of R₂ₐ and R_{2b} is hydrogen and the other is a substituted or unsubstituted alkyl as described herein.

In some of any of the embodiments described herein, R₁ is aminoalkyl or hydroxyalkyl, and in some of these embodiments, each of R₂ₐ and R_{2b} is hydrogen.

Further alternatively, one or both of R₂ₐ and R_{2b} is independently an acyl, as described herein.

In some of any of the embodiments described herein, one or both of R₂ₐ and R_{2b} is independently a substituted or unsubstituted alkyl, as defined herein, or a substituted or unsubstituted cycloalkyl, as defined herein.

In some of any of the embodiments described herein, one or both of R₂ₐ and R_{2b} is independently a substituted or unsubstituted aryl, as defined herein.

In some of any of the embodiments described herein, one or more of R₇-R₉ and of R₁₄ and R₁₅, if present, is an alkyl, a cell-permealizable group as described herein or an acyl such as an amino-substituted alpha-hydroxy acyl.

In some of any of the embodiments described herein one or more of R₇-R₉ and of R₁₄ and R₁₅, if present, is a sulfonyl, for example, an alkyl sulfonyl or an aryl sulfonyl.

Exemplary moieties represented by one or more of R₇-R₉ and of R₁₄ and R₁₅, if present, include, but are not limited to, hydrogen, (R/S)-4-amino-2-hydroxybutyryl (AHB), (R/S)-3-amino-2-hydroxypropionyl (AHP), 5-aminopentanoyl, 5-hydroxypentanoyl, formyl, —C(═O)—O-methyl, -C(═O)—O-ethyl, —C(═O)—O-benzyl, —O-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, -(C H₂)₂NH-ethyl, —(CH₂)₂NH(CH₂)₂NH₂, —(CH₂)₃NH(CH₂)₃NH₂, —(CH₂)₃NH(CH₂)₄NH(CH ₂)₃NH₂, —CH(—NH₂)CH₂(OH), —CH(—OH)CH₂(NH₂), —CH(—OH)—(CH₂)₂(NH₂), —CH(—NH₂) —(CH₂)₂(OH), —CH(—CH₂NH₂)—(CH₂OH), —(CH₂)₄NH(CH₂)₃NH₂, —(CH₂)₂NH(CH₂)₂NH( CH₂)₂NH₂, —(CH₂)₂N(CH₂CH₂NH₂)₂, —CH₂—C(═O)NH₂, —CH(CH₃)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

In some of any of the embodiments described herein, R₇ is hydrogen, (R/S)-4-amino-2-hydroxybutyryl (AHB), (R/S)-3-amino-2-hydroxypropionyl (AHP), 5-aminopentanoyl, 5-hydroxypentanoyl, formyl, —C(═O)—O-methyl, —C(═O)—O-ethyl, —C(═O)—O-benzyl, —O-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, -(C H₂)₂NH-ethyl, —(CH₂)₂NH(CH₂)₂NH₂, —(CH₂)₃NH(CH₂)₃NH₂, —(CH₂)₃NH(CH₂)₄NH(CH ₂)₃NH₂, —CH(—NH₂)CH₂(OH), —CH(—OH)CH₂(NH₂), —CH(—OH)—(CH₂)₂(NH₂), —CH(—NH ₂)—(CH₂)₂(OH), —CH(—CH₂NH₂)—(CH₂OH), —(CH₂)₄NH(CH₂)₃NH₂, —(CH₂)₂NH(CH₂)₂NH (CH₂)₂NH₂, —(CH₂)₂N(CH₂CH₂NH₂)₂, —CH₂—C(═O)NH₂, —CH(CH₂)—C(═O)NH₂, —CH₂-phenyl, —CH(i-propyl)-C(═O)NH₂, —CH(benzyl)-C(═O)NH₂, —(CH₂)₂OH, —(CH₂)₃OH or —CH(CH₂OH)₂.

In some of any of the embodiments described herein, R₇ is other than hydrogen, (R/S)-4-amino-2-hydroxybutyryl (AHB), and (R/S)-3-amino-2-hydroxypropionyl (AHP).

In some of any of the embodiments described herein, R₇ is other than hydrogen, and in some of these embodiments, R₇ is other than an amino-substituted alpha-hydroxy acyl, as defined herein.

In some of any of the embodiments described herein, R₇ is other than alkyl, cycloalkyl, aryl and a cell-permealizable group, as described herein.

In some of any of the embodiments described herein, one or both of R₈ and R₉ is independently hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —COO-methyl, —COO-ethyl, —COO-benzyl, -β-amino-α-hydroxypropionyl, -δ-amino-α-hydroxyvalcryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, -(C H₂)₂NH-ethyl, —(CH₂)₂NH(CH₂)₂NH₂, —(CH₂)₃NH(CH₂)₃NH₂, —(CH₂)₃NH(CH₂)₄NH(CH ₂)₃NH₂, —CH(—NH₂)CH₂(OH), —CH(—OH)CH₂(NH₂), —CH(—OH)—(CH₂)₂(NH₂), —CH(—NH₂) —(CH₂)₂(OH), —CH(—CH₂NH₂)—(CH₂OH), —(CH₂)₄NH(CH₂)₃NH₂, —(CH₂)₂NH(CH₂)₂NH( CH₂)₂NH₂, —(CH₂)₂N(CH₂CH₂NH₂)₂, —CH₂—C(═O)NH₂, —CH(CH₂)—C(═O)NH₂, —CH₂-phenyl, —CH(i-propyl)-C(═O)NH₂, —CH(benzyl)-C(═O)NH₂, —(CH₂)₂OH, —(CH₂)₃OH or —CH(CH₂OH)₂.

In some of any of the embodiments described herein, each of R₇-R₉ is other than hydrogen, (R/S)-4-amino-2-hydroxybutyryl (AHB), and (R/S)-3-amino-2-hydroxypropionyl (AHP).

In some of any of the embodiments described herein, each of R₇-R₉ is other than hydrogen, and in some of these embodiments, each of R₇-R₉ is other than an amino-substituted alpha-hydroxy acyl, as defined herein.

In some of any of the embodiments described herein, each of R₇-R₉ is other than alkyl, cycloalkyl, aryl and a cell-permealizable group, as described herein.

In some of any of the embodiments described herein, an amino-substituted alpha-hydroxy acyl is (S)-4-amino-2-hydroxybutyryl (AHB).

Herein throughout, an amine which bears a substituent other than hydrogen is referred to herein as a “modified amine substituent” or simply as a “modified amine”.

According to some embodiments of the present invention, one or both of the amine substituents at positions 1 (R₇) or 5″ (R₁₂), if present, of the aminoglycoside structure represented by Formulae Ia and Ib, is modified to include a hydrophobic moiety such as alkyl, cycloalkyl, alkaryl and/or aryl, or a group which is positively-charged at physiological pH and which can increase cell permeability of the compound (also referred to herein interchangeably as “cell-permealizable group” or “cell-permealizing group”), such as guanine or guanidine groups, as defined herein, or, alternatively, hydrazine, hidrazide, thiohydrazide, urea and thiourea.

In some of any of the embodiments described herein, none of R₇-R₉ and R₁₄ and R₁₅, if present, is a hydrophobic moiety such as alkyl, cycloalkyl, alkaryl and/or aryl.

In some of any of the embodiments described herein, none of R₇-R₉ and R₁₄ and R₁₅, if present, is a cell-permealizable group, as defined herein.

In some of any of the embodiments described herein, none of R₇-R₉ and R₁₄ and R₁₅, if present, is a modified amine as described herein.

In some of any of the embodiments described herein, one or more R₇-R₉ and R₁₄ and R₁₅, if present, is an acyl, as defined herein, and in some of these embodiments, the acyl can independently be an amino-substituted alpha-hydroxy acyl, as defined herein.

In some of any of the embodiments described herein, whenever a variable is defined as an unsubstituted aryl, the unsubstituted aryl can be, for example, phenyl, 1-anthryl, 1-naphthyl, 2-naphthyl, 2-phenanthryl and/or 9-phenanthryl.

In some of any of the embodiments described herein, whenever a variable is defined as a substituted or unsubstituted heteroaryl, the heteroaryl can be, for example, 2-anthryl, 2-furyl, 2-indolyl, 2-naphthyl, 2-pyridyl, 2-pyrimidyl, 2-pyrryl, 2-quinolyl, 2-thienyl, 3-furyl, 3-indolyl, 3-thienyl, 4-imidazolyl, 4-pyridyl, 4-pyrimidyl, 4-quinolyl, 5-methyl-2-thienyl and/or 6-chloro-3-pyridyl.

In some of any of the embodiments described herein, whenever a variable is defined as a substituted aryl, the aryl can be, for example, 2-(N-ethylamino)phenyl, 2-(N-hexylamino)phenyl, 2-(N-methylamino)phenyl, 2,4-dimethoxyphenyl, 2-acetamidophenyl, 2-aminophenyl, 2-carboxyphenyl, 2-chlorophenyl, 2-ethoxyphenyl, 2-fluorophenyl, 2-hydroxymethylphenyl, 2-hydroxyphenyl, 2-hydroxyphenyl, 2-methoxycarbonylphenyl, 2-methoxyphenyl, 2-methylphenyl, 2-N,N-dimethylaminophenyl, 2-trifluoromethylphenyl, 3-(N,N-dibutylamino)phenyl, 3-(N,N-diethylamino)phenyl, 3,4,5-trimethoxyphenyl, 3,4-dichlorophenyl, 3,4-dimethoxyphenyl, 3,5-dimethoxyphenyl, 3-aminophenyl, 3-biphenylyl, 3-carboxyphenyl, 3-chloro-4-methoxyphenyl, 3-chlorophenyl, 3-ethoxycarbonylphenyl, 3-ethoxyphenyl, 3-fluorophenyl, 3-hydroxymethylphenyl, 3-hydroxyphenyl, 3-isoamyloxyphenyI, 3-isobutoxyphenyl, 3-isopropoxyphenyl, 3-methoxyphenyl, 3-methylphenyl, 3-N,N-dimethylaminophenyl, 3-tolyl, 3-trifluoromethylphenyl, 4-(benzyloxy)phenyl, 4-(isopropoxycarbonyl)phenyl, 4-(N,N-diethylamino)phenyl, 4-(N,N-dihexylamino)phenyl, 4-(N,N-diisopropylamino)phenyl, 4-(N,N-dimethylamino)phenyl, 4-(N,N-di-n-pentylamino)phenyl, 4-(n-hexyloxycarbonyl)phenyl, 4-(N-methylamino)phenyl, 4-(trifluoromethyl)phenyl, 4-aminophenyl, 4-benzyloxyphenyl, 4-biphenylyl, 4-butoxyphenyl, 4-butyramidophenyl, 4-carboxyphenyl, 4-chlorophenyl, 4-ethoxycarbonylphenyl, 4-hexanamidophenyl, 4-hydroxymethylphenyl, 4-hydroxyphenyl, 4-iodophenyl, 4-isobutylphenyl, 4-isobutyramidophenyl, 4-isopropoxyphenyl, 4-isopropylphenyl, 4-methoxyphenyl, 4-methylphenyl, 4-n-hexanamidophenyl, 4-n-hexyloxyphenyl, 4-n-hexylphenyl, 4-nitrophenyl, 4-nitrophenyl, 4-propionamidophenyl, 4-tolyl, 4-trifluoromethylphenyl and/or 4-valeroyloxycarbonylphenyl.

In some of any of the embodiments described herein, the amine substituent at position 1 (R₇, Ring II) in Formula Ia or Ib, is a modified amine, as described herein, such that R₇ is other than hydrogen.

In some of these embodiments, R₇ can be alkyl, cycloalkyl, alkaryl, aryl, an acyl, or an amino-substituted a-hydroxy acyl, as defined herein, such as, for example, (S)-4-amino-2-hydroxybutyryl (AHB), or (S)-4-amino-2-hydroxypropionyl (AHP).

In some of the embodiments where R₇ is alkyl, the alkyl can be, for example, a lower alkyl, of 1-4 carbon atoms, such as, but not limited to, methyl, ethyl, propyl, butyl, isopropyl, and isobutyl, each being optionally substituted, as described herein.

In some of these embodiments, the alkyl is independently a non-substituted alkyl, such as, but not limited to, ethyl, propyl and isopropyl.

In some of these embodiments, the alkyl is independently a substituted methyl, such as, but not limited to, an alkaryl such as benzyl.

Alternatively, R₇ is cycloalkyl, and the cycloalkyl can be, for example, cyclopropyl, cyclobutyl, cyclopentyl and cyclohexyl.

Further alternatively, R₇ is aryl, and the aryl can be, for example, a substituted or unsubstituted phenyl. Non-limiting examples include unsubstituted phenyl and toluene.

In some of any of the embodiments described herein, R₇ is alkyl, cycloalkyl or aryl, as described herein.

In some of these embodiments, R₁ is alkyl, cycloalkyl or aryl, and is preferably alkyl, as defined herein.

In some of these embodiments, R₁ is alkyl, cycloalkyl or aryl, and is preferably alkyl, as defined herein, R₃ is OR₁₆ and R₁₆ is hydrogen (such that R₃ is hydroxy).

In some of any of the embodiments described herein, R₇ is alkyl and in some embodiments it is a lower alkyl, of 1-4 carbon atoms.

In some embodiments, R₇ is an alkyl such as ethyl, propyl, butyl, isopropyl, isobutyl, tert-butyl, each being optionally substituted.

In some embodiments, R₇ is methyl or ethyl, and is preferably a substituted methyl or ethyl. In some of these embodiments, the methyl or ethyl is substituted by, for example, a cycloalkyl or aryl. Such substituents are also referred to in the art as alkylcycloalkyl and alkaryl, respectively. An exemplary alkaryl is benzyl (—CH₂-Phenyl).

In some embodiments, R₇ is propyl or isopropyl.

In some embodiments, R₇ is benzyl.

In some of any of the embodiments described herein, R₇ is a cell-permealizable group, as defined herein, and in some embodiments, R₇ is guanidyl.

In some of any of the embodiments described herein, R₁ is alkyl, cycloalkyl or aryl, and is preferably alkyl, as defined herein, and R₇ is alkyl, as defined herein, preferably, ethyl, propyl, isopropyl or benzyl.

In some of any of the embodiments described herein, R₁ is alkyl, cycloalkyl or aryl, and is preferably alkyl, as defined herein; R₇ is alkyl, as defined herein, preferably, ethyl, propyl, isopropyl or benzyl; and R₃ is hydrogen.

In some of any of the embodiments described herein, R₁ is alkyl, cycloalkyl or aryl, and is preferably alkyl, as defined herein; R₇ is a cell-permealizing group, as defined herein, preferably, guanidine or guanine; and R₃ is hydrogen.

In some of any of the embodiments described herein, R₁ is alkyl, cycloalkyl or aryl, and is preferably alkyl, as defined herein; R₇ is a cell-permealizing group, as defined herein, preferably, guanidine or guanine, more preferably guanidine (guanidinyl).

In some of any of the embodiments described herein, R₇ is hydrogen or a moiety such as (S)-4-amino-2-hydroxybutyryl (AHB), or (S)-4-amino-2-hydroxypropionyl (AHP).

In some of these embodiments, a modified amine is introduced to the compound within a third saccharide moiety (Ring III; e.g., as R₅ in Formula Ia).

Any of the embodiments described herein for Formula Ia, and any combination thereof, are included within the embodiments relating to Formula Ib.

In some of any of the embodiments of Formula Ib, R₁ is alkyl, as defined herein.

In some of any of the embodiments of Formula Ib, R₂ and R₇ are as described in any of the respective embodiments for Formula I.

In some of any of the embodiments of Formula Ib, R₃, R₄ and R₆ are each hydrogen.

In some of these embodiments, R₇ is alkyl, cycloalkyl or aryl, and is preferably alkyl, as described herein.

In some embodiments, R₁ is alkyl, cycloalkyl or aryl, and is preferably alkyl, as defined herein; R₇ is alkyl, as defined herein, preferably, ethyl, propyl, isopropyl or benzyl; and R₅ is a monosaccharide moiety of Formula II, wherein R₁₄ and R₁₅ are both hydrogen.

In some of any of the embodiments of Formula Ib, R₇ is hydrogen, acyl or amino-substituted α-hydroxy-acyl, as defined herein.

In some of these embodiments, one of R₁₄ and R₁₅ is other than hydrogen. In some of these embodiments, one of R₁₄ and R₁₅ is a cell-permealizable group such as, for example, a guanidine group. Alternatively, one of R₁₄ and R₁₅ is alkyl, cycloalkyl or aryl, as defined, for example, for any of the embodiments of R₇.

In some of any of the embodiments described herein, R₁ is alkyl, cycloalkyl or aryl, and is preferably alkyl, as defined herein; R₇ is hydrogen or amino-substituted α-hydroxy-acyl, as defined herein; R₅ is a monosaccharide moiety of Formula II; and R₁₅ is a guanidine group (guanidinyl; guanidyl).

In some of these embodiments, R₁₄ is hydrogen.

In some of any of the embodiments described herein for Formula Ib, R₁₄ is hydrogen or methyl, unless specifically indicated otherwise.

In some of any of the embodiments described herein for Formula Ib, R₁₄ is hydrogen.

In some of any of the embodiments described herein for Formula Ib, R₁₅ is acyl, as defined herein.

In some of any of the embodiments described herein for Formula Ib, one or both of R₁₄ and R₁₅ is a substituted or unsubstituted alkyl, a substituted or unsubstituted cycloalkyl, a substituted or unsubstituted aryl, a substituted or unsubstituted alkaryl, or a substituted or unsubstituted heteroaryl, or an acyl, as these terms are defined herein.

In some of any of the embodiments described herein for Formula Ib, R₁₄ and R₁₅ form together a nitrogen-containing heterocyclic ring, such as, but not limited to, morpholine, piperidine, and piperazine.

In some of any one of the embodiments described herein, and any combination thereof, the stereoconfiguration at position 6′ is an R configuration.

According to some embodiments of the present invention, one or both of the amine substituents at positions 1 or 5″ of the aminoglycoside structure is modified, such that R₇ and/or one or both of R₁₄ and R₁₅ is not hydrogen.

Herein throughout, an amine which bears a substituent other than hydrogen is referred to herein as a “modified amine substituent” or simply as a “modified amine”.

According to some embodiments of the present invention, one or both of the amine substituents at positions 1 or 5″ of the aminoglycoside structure is modified to include a hydrophobic moiety such as alkyl, cycloalkyl, alkaryl and/or aryl, or a group which is positively-charged at physiological pH and which can increase cell permeability of the compound (also referred to herein as “cell-permealizable group”), such as guanine or guanidine groups, as defined herein, or, alternatively, hydrazine, hidrazide, thiohydrazide, urea and thiourea.

According to some of any of the embodiments of the present invention, excluded from the scope of the present invention are compounds known in the art, including any of the documents cited in the Background section of the instant application, which are encompassed by Formula Ia or Ib.

Additional exemplary compounds which are excluded from the scope of the present embodiments include compounds represented by Formula Ia, in which R₂ₐ and R₂ₐ is hydrogen, and R₇ is hydrogen, AHB or AHP, or equivalents of AHB and AHP, as defined in WO 2007/113841 and WO 2012/066546; and compounds represented by Formula Ib, in which each of R₂ₐ and R_{2b} is hydrogen, R₇ is hydrogen, AHB or AHP, or equivalents of AHB and AHP, as defined in WO 2007/113841 and WO 2012/066546, and R₁₄ and R₁₅ are each hydrogen.

According to some embodiments of the present invention, when each of R₂ₐ and R_{2b} is hydrogen, then R₇ is not hydrogen, AHB or AHP, or equivalents of AHB and AHP, as defined in WO 2007/113841 and WO 2012/066546, and/or one or both of R₁₄ and R₁₅, if present, is not hydrogen.

According to some embodiments of the present invention, excluded from the scope of the present embodiments are also compounds represented by Formulae I′a as follows:

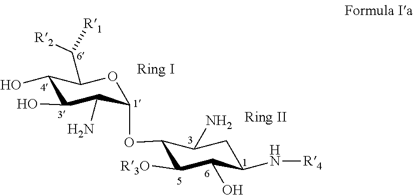

wherein:

the dashed line indicates a stereo-configuration of position 6′ being an R configuration or an S configuration;

R′₁ is alkyl, cycloalkyl, alkaryl or aryl;

R′₂ is NR′R″, wherein R′ and R″ is each independently selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl, as defined herein;

R′₄ is selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl and a cell-permealizable group, such as guanyl or guanidyl; and

R′₃ is hydrogen or a monosaccharide moiety represented by Formula II′:

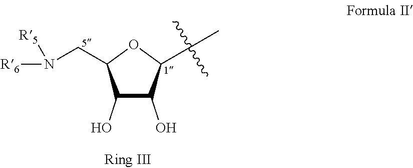

wherein the curved line denotes a position of attachment; and

R′₅ and R′₆ are each independently selected from hydrogen, a substituted or unsubstituted alkyl, a substituted or unsubstituted cycloalkyl, a substituted or unsubstituted aryl, a substituted or unsubstituted alkaryl, a substituted or unsubstituted heteroaryl, acyl, and a cell-permealizable group such as guanyl and guanidinyl, or, alternatively, R′₅ and R′₆ form together a heterocyclic ring,

provided that when R′ and R″ are each hydrogen, R′₄ is not hydrogen, AHB or AHP, and/or at least one of R′₅ and/or R′₆, if present, is not hydrogen.

According to some embodiments of the present invention, excluded from the scope of the present embodiments are also compounds represented by Formulae I′b as follows:

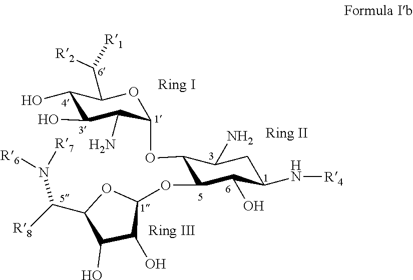

or a pharmaceutically acceptable salt thereof,

wherein:

the dashed line indicates a stereo-configuration of position 6′ being an R configuration or an S configuration;

R′₁ is selected from hydrogen, alkyl, cycloalkyl or aryl;

R₂ is NR′R″, wherein each of R′ and R″ is independently selected from hydrogen, a substituted or unsubstituted alkyl, a substituted or unsubstituted cycloalkyl, a substituted or unsubstituted aryl, a substituted or unsubstituted heteroaryl, a substituted or unsubstituted alkaryl, and an acyl;

R′₄ is selected from hydrogen, acyl, an amino-substituted alpha-hydroxy acyl, a substituted or unsubstituted alkyl, a substituted or unsubstituted cycloalkyl, a substituted or unsubstituted aryl, a substituted or unsubstituted alkaryl, and a cell-permealizable group;

R′₆ and R′₇ are each independently selected from hydrogen, a substituted or unsubstituted alkyl, a substituted or unsubstituted cycloalkyl, a substituted or unsubstituted aryl, a substituted or unsubstituted alkaryl, a substituted or unsubstituted heteroaryl, acyl, and a cell-permealizable group, or, alternatively, R′₅ and R′₆ form together a heterocyclic ring; and

R′₈ is alkyl, cycloalkyl or aryl,

provided that:

when R′ and R″ are each hydrogen, R′₄ is not hydrogen, AHB or AHP, and/or at least one of R′₆ and/or R′₇, if present, is not hydrogen.

For any of the embodiments described herein, and any combination thereof, the compound may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt comprising at least one basic (e.g., amine and/or guanidine) group of the compound which is in a positively charged form (e.g., wherein the basic group is protonated), in combination with at least one counter-ion, derived from the selected base, that forms a pharmaceutically acceptable salt.

The acid addition salts of the compounds described herein may therefore be complexes formed between one or more basic groups of the compound and one or more equivalents of an acid.

Depending on the stoichiometric proportions between the charged group(s) in the compound and the counter-ion in the salt, the acid additions salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

The present embodiments further encompass any enantiomers, diastereomers, prodrugs, solvates, hydrates and/or pharmaceutically acceptable salts of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

According to some of any of the embodiments described herein, a stereo-configuration of each of position 6′ and position 5″ (if present) is independently an R configuration or an S configuration.

According to some of any of the embodiments described herein, a stereo-configuration of position 6′ is an R configuration.

According to some of any of the embodiments described herein, a stereo-configuration of position 5″, if present, is an S configuration.

According to some of any of the embodiments described herein, a stereo-configuration of position 6′ is an R configuration and a stereo-configuration of position 5″, if preset, is an R configuration or an S configuration.

According to some of any of the embodiments described herein, a stereo-configuration of position 6′ is an R configuration and a stereo-configuration of position 5″, if present, is an S configuration.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. A prodrug may also have improved solubility as compared with the parent drug in pharmaceutical compositions. Prodrugs are also often used to achieve a sustained release of the active compound in vivo. An example, without limitation, of a prodrug would be a compound of the present invention, having one or more carboxylic acid moieties, which is administered as an ester (the “prodrug”). Such a prodrug is hydrolyzed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound of the present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The terms “hydroxyl” or “hydroxy”, as used herein, refer to an —OH group.

As used herein, the term “amine” describes a —NR′R″ group where each of R′ and R″ is independently as described herein, and can independently be, for example, hydrogen, alkyl, alkenyl, alkynyl, cycloalkyl, heteroalicyclic, aryl, heteroaryl, alkaryl, alkheteroaryl, or acyl, as these terms are defined herein.

Alternatively, one or both of R′ and R″ can be, for example, hydroxy, alkoxy, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

As used herein, the term “alkyl” describes an aliphatic hydrocarbon including straight chain and branched chain groups. The alkyl may have 1 to 20 carbon atoms, or 1-10 carbon atoms, and may be branched or unbranched. According to some embodiments of the present invention, the alkyl is a low (or lower) alkyl, having 1-4 carbon atoms (namely, methyl, ethyl, propyl and butyl).

Whenever a numerical range; e.g., “1-10”, is stated herein, it implies that the group, in this case the alkyl group, may contain 1 carbon atom, 2 carbon atoms, 3 carbon atoms, etc., up to and including 10 carbon atoms. In some embodiments, the alkyl is a lower alkyl, including 1-6 or 1-4 carbon atoms.

An alkyl can be substituted or unsubstituted. When substituted, the substituent can be, for example, one or more of an alkyl (forming a branched alkyl), an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow. An alkyl substituted by aryl is also referred to herein as “alkaryl”, an example of which is benzyl.

Whenever “alkyl” is described, it can be replaced also by alkenyl or alkynyl. The term “alkyl” as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond, e.g., allyl, vinyl, 3-butenyl, 2-butenyl, 2-hexenyl and i-propenyl. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” refers to an all-carbon monocyclic or fused ring (i.e., rings which share an adjacent pair of carbon atoms), branched or unbranched group containing 3 or more carbon atoms where one or more of the rings does not have a completely conjugated pi-electron system, and may further be substituted or unsubstituted. Exemplary cycloalkyl groups include, for example, cyclopropyl, cyclobutyl, cyclopentyl, cyclohexyl, or cyclododecyl. The cycloalkyl can be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “aryl” describes an all-carbon monocyclic or fused-ring polycyclic (i.e., rings which share adjacent pairs of carbon atoms) groups having a completely conjugated pi-electron system. The aryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. Representative examples are thiadiazole, pyridine, pyrrole, oxazole, indole, purine and the like. The heteroaryl group may be unsubstituted or substituted by one or more substituents. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “heteroalicyclic”, as used herein, describes a monocyclic or fused ring group having in the ring(s) one or more atoms such as nitrogen, oxygen and sulfur. The rings may also have one or more double bonds. However, the rings do not have a completely conjugated pi-electron system. Representative examples are morpholine, piperidine, piperazine, tetrahydrofurane, tetrahydropyrane and the like. The heteroalicyclic may be substituted or unsubstituted. When substituted, the substituent can be, for example, one or more of an alkyl, an alkenyl, an alkynyl, a cycloalkyl, an aryl, a heteroaryl, a heteroalicyclic, a halo, a trihaloalkyl, a hydroxy, an alkoxy and a hydroxyalkyl as these terms are defined hereinbelow.

The term “halide”, as used herein, refers to the anion of a halo atom, i.e. F⁻, Cl⁻, Br⁻ and I⁻.

The term “halo” refers to F, Cl, Br and I atoms as substituents.

The term “alkoxide” refers to an R′—O⁻ anion, wherein R′ is as defined hereinabove.

The term “alkoxy” refers to an —OR′ group, wherein R′ is alkyl or cycloalkyl, as defined herein.

The term “aryloxy” refers to an —OR′ group, wherein R′ is aryl, as defined herein.

The term “heteroaryloxy” refers to an —OR′ group, wherein R′ is heteroaryl, as defined herein.

The term “thioalkoxy” refers to an —SR′ group, wherein R′ is alkyl or cycloalkyl, as defined herein.

The term “thioaryloxy” refers to an —SR′ group, wherein R′ is aryl, as defined herein.

The term “thioheteroaryloxy” refers to an —SR′ group, wherein R′ is heteroaryl, as defined herein.

The term “hydroxyalkyl,” as used herein, refers to an alkyl group, as defined herein, substituted with one or more hydroxy group(s), e.g., hydroxymethyl, 2-hydroxyethyl and 4-hydroxypentyl.

The term “aminoalkyl,” as used herein, refers to an alkyl group, as defined herein, substituted with one or more amino group(s).

The term “alkoxyalkyl,” as used herein, refers to an alkyl group substituted with one or more alkoxy group(s), e.g., methoxymethyl, 2-methoxyethyl, 4-ethoxybutyl, n-propoxyethyl and t-butylethyl.

The term “trihaloalkyl” refers to —CX₃, wherein X is halo, as defined herein. An exemplary haloalkyl is CF₃.

A “guanidine” or “guanidine” or “guanidinyl” or “guanidyl” group refers to an ——RaNC(═NRd)—NRbRc group, where each of Ra, Rb, Rc and Rd can each be as defined herein for R′ and R″.

A “guanyl” or “guanine” group refers to an RaRbNC(═NRd)— group, where Ra, Rb and Rd are each as defined herein for R′ and R″.

In some of any of the embodiments described herein, the guanidine group is —NH—C(═NH)—NH₂.

In some of any of the embodiments described herein, the guanyl group is H₂N—C(═NH)— group.

Any one of the amine (including modified amine), guanidine and guanine groups described herein is presented as a free base form thereof, but is meant to encompass an ionized form thereof at physiological pH, and/or within a salt thereof, e.g., a pharmaceutically acceptable salt thereof, as described herein.

Whenever an alkyl, cycloalkyl, aryl, alkaryl, heteroaryl, heteroalicyclic, acyl and any other moiety as described herein is substituted, it includes one or more substituents, each can independently be, but are not limited to, hydroxy, alkoxy, thiohydroxy, thioalkoxy, aryloxy, thioaryloxy, alkaryl, alkenyl, alkynyl, sulfonate, sulfoxide, thiosulfate, sulfate, sulfite, thiosulfite, phosphonate, cyano, nitro, azo, sulfonamide, carbonyl, thiocarbonyl, C-carboxylate, O-carboxylate, N-thiocarbamate, O-thiocarbamate, oxo, thiooxo, oxime, acyl, acyl halide, azo, azide, urea, thiourea, N-carbamate, O-carbamate, C-amide, N-amide, guanyl, guanidyl, hydrazine and hydrazide, as these terms are defined herein.

The term “cyano” describes a —C≡N group.

The term “nitro” describes an —NO₂ group.

The term “sulfate” describes a —O—S(═O)₂—OR′ end group, as this term is defined hereinabove, or an —O—S(═O)₂—O— linking group, as these phrases are defined hereinabove, where R′ is as defined hereinabove.

The term “thiosulfate” describes a —O—S(═S)(═O)—OR′ end group or a —O—S(═S)(═O)—O— inking group, as these phrases are defined hereinabove, where R′ is as defined hereinabove.

The term “sulfite” describes an —O—S(═O)—O—R′ end group or a —O—S(═O)—O— group linking group, as these phrases are defined hereinabove, where R′ is as defined hereinabove.

The term “thiosulfite” describes a —O—S(═S)—O—R′ end group or an —O—S(═S)—O— group linking group, as these phrases are defined hereinabove, where R′ is as defined hereinabove.

The term “sulfinate” describes a —S(═O)—OR′ end group or an —S(═O)—O— group linking group, as these phrases are defined hereinabove, where R′ is as defined hereinabove.

The term “sulfoxide” or “sulfinyl” describes a —S(═O)R′ end group or an —S(═O)— linking group, as these phrases are defined hereinabove, where R′ is as defined hereinabove.

The term “sulfonate” or “sulfonyl” describes a —S(═O)₂—R′ end group or an —S(═O)₂— linking group, as these phrases are defined hereinabove, where R′ is as defined herein.

The term “S-sulfonamide” describes a —S(═O)₂—NR′R″ end group or a —S(═O)₂—NR′— linking group, as these phrases are defined hereinabove, with R′ and R″ as defined herein.

The term “N-sulfonamide” describes an R′S(═O)₂—NR″— end group or a —S(═O)₂—NR′— linking group, as these phrases are defined hereinabove, where R′ and R″ are as defined herein.

The term “carbonyl” or “carbonate” as used herein, describes a —C(═O)—R′ end group or a —C(═O)— linking group, as these phrases are defined hereinabove, with R′ as defined herein.

The term “thiocarbonyl ” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “acyl halide” describes a —(C═O)R″″ group wherein R″″ is halide, as defined hereinabove.

The term “azo” or “diazo” describes an —N═NR′ end group or an —N═N— linking group, as these phrases are defined hereinabove, with R′ as defined hereinabove.

The term “azide” describes an —N₃ end group.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″— end group or a R′C(═O)—N— linking group, as these phrases are defined hereinabove, where R′ and R″ are as defined herein.

The term “hydrazine” describes a —NR′—NR″R′″ end group or a —NR′—NR″— linking group, as these phrases are defined hereinabove, with R′, R″, and R′″ as defined herein.

As used herein, the term “hydrazide” describes a —C(═O)—NR′—NR″R′″ end group or a —C(═O)—NR′—NR″— linking group, as these phrases are defined hereinabove, where R′, R″ and R′″ are as defined herein.

As used herein, the term “thiohydrazide” describes a —C(═S)—NR′—NR″R′″ end group or a —C(═S)—NR′—NR″— linking group, as these phrases are defined hereinabove, where R′, R″ and R′″ are as defined herein.

Further according to embodiments of the present invention, there are provided processes of preparing the compounds as described herein.

The processes of preparing pseudo-trisaccharide compounds according to some embodiments of the present invention are generally effected by devising appropriate acceptor aminoglycoside acceptor molecules and corresponding donor molecules, as is known in the art of aminoglycosides.

Generally, the synthesis of pseudo-trisaccharide compounds according to some embodiments of the present invention is accomplished using suitable acceptor and donor molecules and reaction conditions which allow reacting the donor and acceptor and removing the protecting group so as to obtain a desired pseudo-trisaccharide of Formula Ia.

The term “acceptor” is used herein to describe the skeletal structure derived from paromamine which has an available (unprotected) hydroxyl group at position C5, which is reactive during a glycosylation reaction, and can accept a glycosyl.

The term “donor” is used herein to describe the glycosyl that reacts with the acceptor to form the final pseudo-trisaccharide compound.

The term “glycosyl”, as used herein, refers to a chemical group which is obtained by removing the hydroxyl group from the hemiacetal function of a monosaccharide.

The donors and acceptors are designed so as to form the desired compounds according to some embodiments of the present invention. The following describes some embodiments of this aspect of the present invention, presenting exemplary processes of preparing exemplary subsets of the compounds described herein. More detailed processes of preparing exemplary compounds according to some embodiments of the present invention, are presented in the Examples section that follows below.

The syntheses of the compounds according to some embodiments of the present invention, generally include (i) preparing an acceptor compound by selective protection of one or more hydroxyls and amines at selected positions present on the paromamine scaffold, leaving the selected position (e.g., C5) unprotected and therefore free to accept a donor (glycosyl) compound as defined herein; (ii) preparing a donor compound by selective protection of one or more hydroxyls and amines at selected positions present on the glycosyl, leaving one position unprotected and therefore free to couple with an acceptor compound as defined herein; (iii) subjecting the donor and the acceptor to a coupling reaction; and (iii) removing the protecting groups to thereby obtain the desired compound.

The phrase “protected group”, as used herein, refers to a group that is substituted or modified so as to block its functionality and protect it from reacting with other groups under the reaction conditions (e.g., a coupling reaction as described herein). A protected group is re-generated by removal of the substituent or by being re-modified.

When an “amino-protected group” or “hydroxyl-protected group” is used, it is meant that a protecting group is attached or used to modify the respective group so as to generate the protected group.

The phrase “protecting group”, as used herein, refers to a substituent or a modification that is commonly employed to block or protect a particular functionality while reacting other functional groups on the compound. The protecting group is selected so as to release the substituent or to be re-modified, to thereby generate the desired unprotected group.

For example, an “amino-protecting group” or “amine-protecting group” is a substituent attached to an amino group, or a modification of an amino group, that blocks or protects the amino functionality in the compound, and prevents it from participating in chemical reactions. The amino-protecting group is removed by removal of the substituent or by a modification that re-generates an amine group.

Suitable amino-protected groups include azide (azido), N-phthalimido, N-acetyl, N-trifluoroacetyl, N-t-butoxycarbonyl (BOC), N-benzyloxycarbonyl (CBz) and N-9-fluorenylmethylenoxycarbonyl (Fmoc).

A “hydroxyl-protecting group” or “hydroxyl-protecting group” refers to a substituent or a modification of a hydroxyl group that blocks or protects the hydroxyl functionality, and prevents it from participating in chemical reactions. The hydroxy-protecting group is removed by removal of the substituent or by a modification that re-generates a hydroxy group.

Suitable hydroxy protected groups include isopropylidene ketal and cyclohexanone dimethyl ketal (forming a 1,3-dioxane with two adjacent hydroxyl groups), 4-methoxy-1-methylbenzene (forming a 1,3-dioxane with two adjacent hydroxyl groups), O-acetyl, O-chloroacetyl, O-benzoyl and O-silyl.

For a general description of protecting groups and their use, see T. W. Greene, Protective Groups in Organic Synthesis, John Wiley & Sons, New York, 1991.

According to some embodiments, the amino-protected groups include an azido (N₃-) and/or an N-phthalimido group, and the hydroxyl-protecting groups include O-acetyl (AcO-), O-benzoyl (BzO-) and/or O-chloroacetyl.

It is noted herein that when applicable, a “protected group” refers to a moiety in which one reactive function on a compound is protected or more than one function are protected at the same time, such as in the case of two adjacent functionalities, e.g., two hydroxyl groups that can be protected at once by a isopropylidene ketal.

In some embodiments, the donor compound is a protected monosaccharide which can be represented by the general Formula III.

In some embodiments, the donor compound is a protected monosaccharide which can be represented by the general Formula III, having a leaving group at position 1″ thereof, denoted L, and optionally a substituent R₁₂ at position 5″, as defined herein:

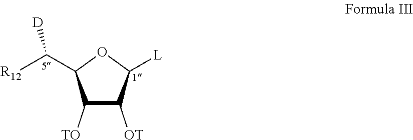

wherein:

L is a leaving group;

OT is a donor protected hydroxyl group;

R₁₂ is as defined herein for Formula Ib (the configuration at the 5″ position as presented in Formula III being a non-limiting example); and

D is a protected or unprotected form of NR₁₄R₁₅ as defined for Formula Ib, wherein when R₁₄ and R₁₅ are both hydrogen, D is a donor protected amine group.

As used herein, the phrase “leaving group” describes a labile atom, group or chemical moiety that readily undergoes detachment from an organic molecule during a chemical reaction, while the detachment is typically facilitated by the relative stability of the leaving atom, group or moiety thereupon. Typically, any group that is the conjugate base of a strong acid can act as a leaving group. Representative examples of suitable leaving groups according to some of the present embodiments include, without limitation, trichloroacetimidate, acetate, tosylate, triflate, sulfonate, azide, halide, hydroxy, thiohydroxy, alkoxy, cyanate, thiocyanate, nitro and cyano.

According to some embodiments of the present invention, each of the donor hydroxyl-protected groups is O-benzoyl and the donor amino-protected group in either R15 or R₁₄ is azido, although other protecting groups are contemplated.

It is to be noted that when one of R₁₄ and R₁₅ is other than hydrogen, it can be protected or unprotected. Typically, when one of R₁₄ and R₁₅ is guanine or guanidine, a protecting group suitable for the reaction conditions (e.g., of a coupling reaction with an acceptor) can be used. Optionally, the guanine or guanidine is unprotected. When one of R₁₄ and R₁₅ is an alkyl, aryl or cycloalkyl, typically D in Formula III is an unprotected form of NR₁₄R₁₅.

The structure of the donor compound sets the absolute structure of Ring III in the resulting compound according to some embodiments of the present invention, namely the stereo-configuration of the 5″ position and the type of R₁₄, R₁₅ and R₁₂ in Formula Ib.

Exemplary acceptor molecules suitable for use in the preparation of the compounds described herein, are represented by Formula IV:

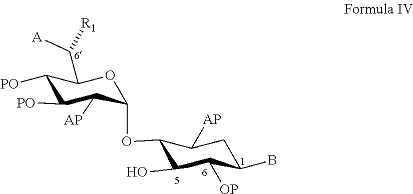

wherein:

the dashed line represents an S-configuration or an R-configuration at position 6′;

OP is an acceptor protected hydroxyl group;

AP is an acceptor protected amine group;

R₁ is as defined herein for Formula Ia or Ib;

A is an acceptor protected amine group (AP); or can otherwise be one of the other groups defining NR₂ₐR_{2b}, either protected or unprotected, according to the chemical nature of these groups and the reaction conditions; and

B is an acceptor protected amine group, in case R₇ is Formula Ia is hydrogen, or can otherwise be a protected or unprotected form of the groups defining R₇.

According to some embodiments of the present invention, the acceptor hydroxyl-protected group is O-acetyl.

According to some embodiments of the present invention, the donor amino-protected group is azido, although other protecting groups are contemplated.

The acceptor hydroxyl-protected groups and the acceptor amino-protected groups at the various positions of the acceptors can be the same or different each position.

In some embodiments, for example, in case R₇ is other than H, the acceptor is prepared by generating the moiety B, prior to reacting it with the donor.

The structure of the acceptor compound sets the absolute structure of Ring I and Ring II in the resulting compound according to some embodiments of the present invention.

In some embodiments, the synthesis of pseudo-disaccharide compounds of Formula Ia, according to some embodiments of the present invention, is accomplished using an amino-protected compound of Formula V:

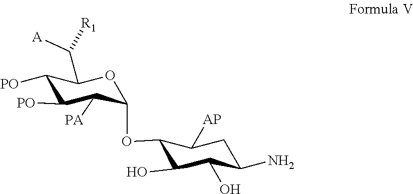

wherein:

the dashed line represents an S-configuration or an R-configuration at position 6′;

AP is an acceptor protected amine group;

R₁ is as defined herein for Formula Ia;

A is an acceptor protected amine group (AP), as described herein; or can otherwise be one of the other groups defining NR₂ₐR_{2b}, either protected or unprotected, according to the chemical nature of these groups and the reaction conditions.

Therapeutic Uses:

The compounds according to some embodiments of the present invention are effective in treating medical conditions associated with a pathogenic microorganism in a subject.

The compounds presented herein can also be effective in treating medical conditions associated with pathogenic microorganisms which have already developed resistance to any antibiotic agent.

The phrases “effective in treating medical conditions associated with pathogenic microorganisms”, “effective in treating a subject diagnosed with a medical conditions associated with pathogenic microorganisms” and/or “for use in the treatment of a medical condition associated with a pathogenic microorganism in a subject”, as used herein interchangeably, refer to characteristics of a substance, such as the compounds according to some embodiments of the present invention, that can effect death, killing, eradication, elimination, reduction in number, reduction of growth rate, reduction of a load, and/or a change in population distribution of one or more species of pathogenic microorganisms, as well as effecting a reduction or prevention of the emergence of resistance of such microorganisms to the substance.

Herein throughout, the phrase “pathogenic microorganism” is used to describe any microorganism which can cause a disease or disorder in a higher organism, such as mammals in general and a human in particular. The pathogenic microorganism may belong to any family of organisms such as, but not limited to prokaryotic organisms, eubacterium, archaebacterium, eukaryotic organisms, yeast, fungi, algae, protozoan, and other parasites.

Non-limiting examples of pathogenic microorganism include Plasmodium falciparum and related malaria-causing protozoan parasites, Acanthamoeba and other free-living amoebae, Aeromonas hydrophila, Anisakis and related worms, and further include, but not limited to Acinetobacter baumanii, Ascaris lumbricoides, Bacillus cereus, Brevundimonas diminuta, Campylobacter jejuni, Clostridium botulinum, Clostridium perfringens, Cryptosporidium parvum, Cyclospora cayetanensis, Diphyllobothrium, Entamoeba histolytica, certain strains of Escherichia coli, Eustrongylides, Giardia lamblia, Klebsiella pneumoniae, Listeria monocytogenes, Nanophyetus, Plesiomonas shigelloides, Proteus mirabilis, Pseudomonas aeruginosa, Salmonella, Serratia odorifera, Shigella, Staphylococcus aureus, Stenotrophomonas maltophilia, Streptococcus, Trichuris trichiura, Vibrio cholerae, Vibrio parahaemolyticus, Vibrio vulnificus and other vibrios, Yersinia enterocolitica, Yersinia pseudotuberculosis and Yersinia kristensenii.

Other pathogens include Strep. pyogenes (Group A), Strep. pneumoniae, Strep. GpB, Strep. viridans, Strep. GpD (Enterococcus), Strep. GpC and GpG, Staph. aureus, Staph. epidermidis, Bacillus subtilis, Bacillus anthracis, Listeria monocytogenes, Anaerobic cocci, Clostridium spp., Actinomyces spp, Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aeruginosa, Campylobacter jejuni, Vibrio parahaemolyticus, Brucella spp., Neisseria meningitidis, Neisseria gonorrhoea, Bacteroides fragilis, Fusobacterium spp., Mycobacterium tuberculosis (including MDR and XDR strains from hospital origins isolated from patients) and mycobacterium smegmatis.

Accordingly, a condition associated with a pathogenic microorganism describes an infectious condition that results from the presence of the microorganism in a subject. The infectious condition can be, for example, a bacterial infection, a fungal infection, a protozoal infection, and the like, collectively referred to herein as “microbial infection”.

Some higher forms of microorganisms are pathogenic per-se, and other harbor lower forms of pathogenic bacteria, thus present a medical threat expressed in many medical conditions, such as, without limitation, actinomycosis, anthrax, aspergillosis, bacteremia, bacterial skin diseases, bartonella infections, botulism, brucellosis, burkholderia infections, campylobacter infections, candidiasis, cat-scratch disease, chlamydia infections, cholera, clostridium infections, coccidioidomycosis, cryptococcosis, dermatomycoses, dermatomycoses, diphtheria, ehrlichiosis, epidemic louse borne typhus, Escherichia coli infections, fusobacterium infections, gangrene, general infections, general mycoses, gram-negative bacterial infections, Gram-positive bacterial infections, histoplasmosis, impetigo, klebsiella infections, legionellosis, leprosy, leptospirosis, listeria infections, lyme disease, maduromycosis, melioidosis, mycobacterium infections, mycoplasma infections, necrotizing fasciitis, nocardia infections, onychomycosis, ornithosis, pneumococcal infections, pneumonia, pseudomonas infections, Q fever, rat-bite fever, relapsing fever, rheumatic fever, rickettsia infections, Rocky-mountain spotted fever, salmonella infections, scarlet fever, scrub typhus, sepsis, sexually transmitted bacterial diseases, staphylococcal infections, streptococcal infections, surgical site infection, tetanus, tick-borne diseases, tuberculosis, tularemia, typhoid fever, urinary tract infection, vibrio infections, yaws, yersinia infections, Yersinia pestis plague, zoonoses and zygomycosis.

The compounds presented herein can be effectively used against bacterial strains which have developed or are prone to or capable of developing resistance to at least one antimicrobial strain. Non-limiting examples of such bacterial strains include:

(a) Gram-positive bacteria such as Strep. pyogenes (Group A), Strep. pneumoniae, Strep. GpB, Strep. viridans, Strep. GpD -(Enterococcus), Strep. GpC and GpG, Staph. aureus, Staph. epidermidis, Bacillus subtilis, Bacillus anthraxis, Listeria monocytogenes, Anaerobic cocci, Clostridium spp., and Actinomyces spp; and

(b) Gram-negative bacteria such as Escherichia coli, Enterobacter aerogenes, Kiebsiella pneumoniae, Proteus mirabilis, Proteus vulgaris, Morganella morganii, Providencia stuartii, Serratia marcescens, Citrobacter freundii, Salmonella typhi, Salmonella paratyphi, Salmonella typhi murium, Salmonella virchow, Shigella spp., Yersinia enterocolitica, Acinetobacter calcoaceticus, Flavobacterium spp., Haemophilus influenzae, Pseudomonas aueroginosa, Campylobacter jejuni, Vibrio parahaemolyticus, Brucella spp., Neisseria meningitidis, Neisseria gonorrhoea, Bacteroides fragilis, and Fusobacterium spp.

According to some embodiments of the present invention, the compounds presented herein can be effectively used against bacterial strains which have developed or are prone to or capable of developing resistance to at least one antimicrobial strain, such as, but not limited to, E.coli R477-100, E.coli ATCC 25922, E.coli AG100B, E.coli AG100A, B. subtilis ATCC 6633, MRSA ATCC 43300 and E.coli ATCC 35218.

Thus, according to one aspect of the present invention there is provided a method of treating a medical condition associated with a pathogenic microorganism in a subject. The method is effected by administering to that subject, a therapeutically effective amount of a compound as presented herein.

As used herein, the phrase “therapeutically effective amount” describes an amount of an active agent being administered, which will relieve to some extent one or more of the symptoms of the condition being treated. In the context of the present embodiments, the phrase “therapeutically effective amount” describes an amount of a compound being administered and/or re-administered, which will relieve to some extent one or more of the symptoms of the condition being treated by being at a level that is harmful to the target microorganism(s), and cause a disruption to the life-cycle of the target microorganism(s), namely a bactericidal level or otherwise a level that inhibits the microorganism growth or eradicates the microorganism.

The efficacy of any antimicrobial agent, including the compounds presented herein, is oftentimes referred to in minimal inhibitory concentration units, or MIC units. A MIC is the lowest concentration of an antimicrobial agent, typically measured in micro-molar (μM) or micrograms per milliliter (μg/ml) units, which can inhibit the growth of a microorganism after a period of incubation, typically 24 hours. MIC values are used as diagnostic criteria to evaluate resistance of microorganisms to an antimicrobial agent, and for monitoring the activity of an antimicrobial agent in question. MICs are determined by standard laboratory methods, as these are described and demonstrated in the Examples section that follows. Standard laboratory methods typically follow a standard guideline of a reference body such as the Clinical and Laboratory Standards Institute (CLSI), British Society for Antimicrobial Chemotherapy (BSAC) or The European Committee on Antimicrobial Susceptibility Testing (EUCAST). In clinical practice, the minimum inhibitory concentrations are used to determine the amount of antibiotic agent that the subject receives as well as the type of antibiotic agent to be used.

According to another aspect of embodiments of the present invention, each of the compounds described herein is for use in treating a medical condition associated with a pathogenic microorganism and/or in treating a subject diagnosed with a medical condition associated with a pathogenic microorganism.

According to another aspect of embodiments of the present invention, there is provided a use of any of the compounds described herein as a medicament or in the manufacture of a medicament. In some embodiments, the medicament is for treating a medical condition associated with a pathogenic microorganism and/or a subject diagnosed with a medical condition associated with a pathogenic microorganism.

The compounds presented herein can be administered via any administration route, including, but not limited to, orally, by inhalation, or parenterally, for example, by intravenous drip or intraperitoneal, subcutaneous, intramuscular or intravenous injection, or topically (including ophtalmically, vaginally, rectally, intranasally).

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the phrase “therapeutically effective amount” describes an amount of the polymer being administered which will relieve to some extent one or more of the symptoms of the condition being treated.

Pharmaceutical Compositions:

In any of the methods and uses described herein, the compounds described herein can be utilized either per se or form a part of a pharmaceutical composition, which further comprises a pharmaceutically acceptable carrier, as defined herein.

According to an aspect of some embodiments of the present invention, there is provided a pharmaceutical composition which comprises, as an active ingredient, any of the novel compounds described herein and a pharmaceutically acceptable carrier.

As used herein a “pharmaceutical composition” refers to a preparation of the compounds presented herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, PA, latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the compounds presented herein into preparations which, can be used pharmaceutically. Proper formulation is dependent upon the route of administration chosen.

According to some embodiments, the administration is effected orally. For oral administration, the compounds presented herein can be formulated readily by combining the compounds with pharmaceutically acceptable carriers well known in the art. Such carriers enable the compounds presented herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the compounds presented herein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For injection, the compounds presented herein may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active aminoglycoside compounds doses.

For buccal administration, the compositions may take the form of tablets or lozenges formulated in conventional manner.

For administration by inhalation, the compounds presented herein are conveniently delivered in the form of an aerosol spray presentation (which typically includes powdered, liquefied and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compounds presented herein and a suitable powder base such as, but not limited to, lactose or starch.

The compounds presented herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the compounds preparation in water-soluble form. Additionally, suspensions of the compounds presented herein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the compounds presented herein to allow for the preparation of highly concentrated solutions.

Alternatively, the compounds presented herein may be in powder form for constitution with a suitable vehicle, e.g., sterile, pyrogen-free water, before use.

The compounds presented herein may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of compounds presented herein effective to prevent, alleviate or ameliorate symptoms of the disorder, or prolong the survival of the subject being treated.

Determination of a therapeutically effective amount is well within the capability of those skilled in the art, especially in light of the detailed disclosure provided herein.

For any compounds presented herein used in the methods of the present embodiments, the therapeutically effective amount or dose can be estimated initially from activity assays in animals. For example, a dose can be formulated in animal models to achieve a circulating concentration range that includes the mutation suppression levels as determined by activity assays (e.g., the concentration of the test compounds which achieves a substantial read-through of the truncation mutation). Such information can be used to more accurately determine useful doses in humans.

Toxicity and therapeutic efficacy of the compounds presented herein can be determined by standard pharmaceutical procedures in experimental animals, e.g., by determining the EC₅₀ (the concentration of a compound where 50% of its maximal effect is observed) and the LD₅₀ (lethal dose causing death in 50% of the tested animals) for a subject compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide plasma levels of the compounds presented herein which are sufficient to maintain the desired effects, termed the minimal effective concentration (MEC). The MEC will vary for each preparation, but can be estimated from in vitro data; e.g., the concentration of the compounds necessary to achieve 50-90% expression of the whole gene having a truncation mutation, i.e. read-through of the mutation codon. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the chronic condition to be treated, dosing can also be a single periodic administration of a slow release composition described hereinabove, with course of periodic treatment lasting from several days to several weeks or until sufficient amelioration is effected during the periodic treatment or substantial diminution of the disorder state is achieved for the periodic treatment.

The amount of a composition to be administered will, of course, be dependent on the subject being treated, the severity of the affliction, the manner of administration, the judgment of the prescribing physician, etc. Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a compound according to the present embodiments, formulated in a compatible pharmaceutical carrier may also be prepared, placed in an appropriate container, and labeled for treatment of an indicated condition or diagnosis, as is detailed hereinabove.

Thus, in some embodiments, the pharmaceutical composition is packaged in a packaging material and identified in print, in or on the packaging material, for use in the treatment of a medical condition associated with a pathogenic microorganism, as defined herein.

In any of the composition, methods and uses described herein, the compounds can be utilized in combination with other agents useful in the treatment of the medical conditions described herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is expected that during the life of a patent maturing from this application many relevant genetic diseases and disorders as defined herein will be uncovered and the scope of this term is intended to include all such new disorders and diseases a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-49. (canceled)

50. A compound represented by Formula I: 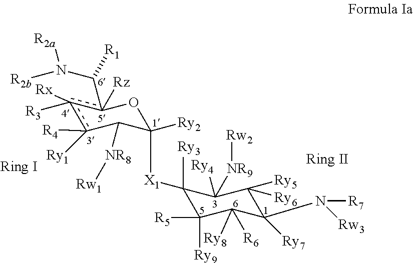
or a pharmaceutically acceptable salt thereof,
wherein:
the dashed line indicates a stereo-configuration of position 6′ being an R configuration or an S configuration;
X₁ is O or S;
the dashed bond between C4′ and C5′ in Ring I represents a single bond or a double bond;
the dashed bond between C4′ and C3′ in Ring I represents a single bond or a double bond;
Rx, Ry1 and Rz are each independently hydrogen, alkyl or cycloalkyl, or absent, wherein Rx and Rz are both absent in case the dashed bond between C4′ and C5′ is a double bond, and Rx and Ry1 are both absent in case the dashed bond between C4′ and C3′ is a double bond;
Ry2-Ry9 and Rw1-Rw3 are each independently selected from hydrogen, alkyl, alkenyl, alkynyl, aryl, heteroaryl and cycloalkyl, each being substituted or unsubstituted, or, alternatively, each can be as defined herein for R₇-R₉;
R₁ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated and/or substituted or unsubstituted hydroxy alkyl (e.g., —CH₂—OH);
R₂ₐ and R_{2b} are each independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted heteroalicyclic and acyl;
R₃-R₆ are each independently selected from the group consisting of hydrogen, alkyl, alkenyl, alkynyl, cycloalkyl, heteroalicyclic, aryl, heteroaryl, amine and OR₁₆, wherein R₁₆ is independently selected from a monosaccharide moiety, an oligosaccharide moiety, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl and acyl; and
R₇-R₉ are each independently selected from the group consisting of hydrogen, acyl, an amino-substituted alpha-hydroxy acyl, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted alkaryl, carboxylate, sulfonyl (including alkyl sulfonyl and aryl sulfonyl) and a cell-permealizable group.

51. The compound of claim 50, wherein at least one of R₃-R₆ is OR₁₆.

52. The compound of claim 51, wherein R₁₆ is an aryl or a heteroaryl.

53. The compound of claim 50, wherein at least one of R₃-R₆ is independently selected from the group consisting of 2-anthryloxy, 2-furyloxy, 2-indolyloxy, 2-naphthyloxy, 2-pyridyloxy, 2-pyrimidyloxy, 2-pyrryloxy, 2-quinolyloxy, 2-thienyloxy, 3-furyloxy, 3-indolyloxy, 3-thienyloxy, 4-imidazolyloxy, 4-pyridyloxy, 4-pyrimidyloxy, 4-quinolyloxy, 5-methyl-2-thienyloxy and 6-chloro-3-pyridyloxy.

54. The compound of claim 50, wherein R₃ is OR₁₆ and R₁₆ is selected from the group consisting of hydrogen, methyl, ethyl, propyl, butyl, pentyl, propenyl, 2-hydroxyethyl, 3-hydroxypropyl, 2,3-dihydroxypropyl and methoxymethyl.

55. The compound of claim 50, wherein at least one of R₃-R₆ is OR₁₆ and R₁₆ is independently an acyl.

56. The compound of claim 50, wherein at least one of R₃-R₆ is OR₁₆ in which R₁₆ is said monosaccharide moiety.

57. The compound of claim 56, wherein said monosaccharide moiety is represented by Formula II: 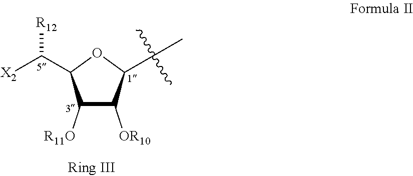
wherein:
the curved line denotes a position of attachment;
the dashed line indicates a stereo-configuration of position 5″ being an R configuration or an S configuration;
X₂ is OR₁₃ or NR₁₄R₁₅;
each of R₁₀, R₁₁ and R₁₃ is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, and acyl;
R₁₂ is selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, a substituted or unsubstituted amine, a substituted or unsubstituted amide, an acyl, a carboxylate, and a saturated or unsaturated and/or substituted or unsubstituted hydroxyalkyl;
each of R₁₄ and R₁₅ is independently selected from the group consisting of hydrogen, a substituted or unsubstituted alkyl, a substituted or unsubstituted alkenyl, a substituted or unsubstituted alkynyl, a substituted or unsubstituted cycloalkyl, a substituted or unsubstituted aryl, a substituted or unsubstituted heteroaryl, a substituted or unsubstituted alkaryl, acyl, and a cell-permealizable group, or, alternatively, R₁₄ and R₁₅, when present, form together a heterocyclic ring.

58. The compound of claim 57, being represented by Formula Ib:

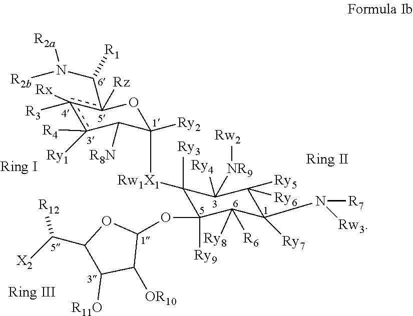

59. The compound of claim 57, wherein at least one of R₃-R₆ is OR₁₆ and R₁₆ is independently an acyl.

60. The compound of claim 57, wherein R₁₂ is other than hydrogen.

61. The compound of claim 57, wherein at least one of R₁₀, R₁₁ and R₁₃ if present is an acyl.

62. The compound of claim 59, wherein at least one of R₁₀, R₁₁ and R₁₃ if present is an acyl.

63. The compound of claim 60, wherein at least one of R₁₀, R₁₁ and R₁₃ if present is an acyl.

64. The compound of claim 50, wherein X₁ is O.

65. The compound of claim 50, wherein the bond between C4′ and C5′ in Ring I is a single bond.

66. The compound of claim 50, wherein the bond between C4′ and C5′ in Ring I is a double bond and Rx is absent.

67. The compound of claim 50, wherein R₁ is other than hydrogen.

68. The compound of claim 67, wherein R₁ is a hydroxyalkyl.

69. The compound of claim 67, wherein R₁ is a substituted or unsubstituted alkyl, a substituted or unsubstituted alkenyl or a substituted or unsubstituted alkynyl.

70. The compound of claim 67, wherein R₁ is an aryl.

71. The compound of claim 67, wherein R₁ is a substituted or unsubstituted heteroaryl.

72. The compound of claim 67, wherein R₁ is a substituted aryl.

73. The compound of claim 67, wherein R₁ is amine.

74. The compound of claim 50, wherein each of R₂ₐ and R_{2b} is hydrogen.

75. The compound of claim 50, wherein each of R₇, R₈ and R₉ is independently selected from the group consisting of hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(CH₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(—NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂—C(═O)NH₂, —CH(CH₂)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

76. The compound of claim 58, wherein each of R₇, R₈ and R₉ is independently is selected from the group consisting of hydrogen, (R/S)-4-amino-2-hydroxybutyryl (AHB), (R/S)-3-amino-2-hydroxypropionate (AHP), (R/S)-3-amino-2-hydroxypropionyl, 5-aminopentanoyl, 5-hydroxypentanoyl, formyl, —C(═O)—O-methyl, —C(═O)—O-ethyl, —C(═O)—O-benzyl, β-amino-α-hydroxypropionyl, -δ-amino-α-hydroxyvaleryl, -β-benzyloxycarbonylamino-α-hydroxypropionyl, -δ-benzyloxycarbonylamino-α-hydroxyvaleryl, methylsulfonyl, phenylsulfonyl, benzoyl, propyl, isopropyl, —(CH₂)₂NH₂, —(CH₂)₃NH₂, —CH₂CH(NH₂)CH₃, —(CH₂)₄NH₂, —(CH₂)₅NH₂, —(CH₂)₂NH-ethyl, —(CH₂)₂NH(CH₂)₂NH₂, —(CH₂)₃NH(CH₂)₃NH₂, —(CH₂)₃NH(CH₂)₄NH(CH₂)₃NH₂, —CH(—NH₂)CH₂(OH), —CH(—OH)CH₂(NH₂), —CH(—OH)—(CH₂)₂(NH₂), —CH(—NH₂)—(CH₂)₂(OH), —CH(—CH₂NH₂)—(CH₂OH), —(CH₂)₄NH(CH₂)₃NH₂, —(CH₂)₂NH(CH₂)₂NH(CH₂)₂NH₂, —(CH₂)₂N(CH₂CH₂NH₂)₂, —CH₂—C(═O)NH₂, —CH(CH₂)—C(═O)NH₂, —CH₂-phenyl, —CH(i-propyl)-C(═O)NH₂, —CH(benzyl)-C(═O)NH₂, —(CH₂)₂OH, —(CH₂)₃OH and —CH(CH₂OH)₂.

77. The compound of claim 50, wherein said acyl is selected from the group consisting of a hydrocarbon acyl radical having from 2 to 18 carbon atoms, optionally substituted by one or more of halo, nitro, hydroxy, amine, cyano, thiocyano, and alkoxy.

78. The compound of claim 50, wherein said acyl is derived from an acid selected from the group consisting of a saturated or unsaturated and/or substituted or unsubstituted aliphatic carboxylic acid, acetic acid, propionic acid, butyric acid, isobutyric acid, tert-butylacetic acid, valeric acid, isovaleric acid, caproic acid, caprylic acid, decanoic acid, dodecanoic acid, lauric acid, tridecanoic acid, myristic acid, pentadecanoic acid, palmitic acid, margaric acid, stearic acid, acrylic acid, crotonic acid, undecylenic acid, oleic acid, hexynoic acid, heptynoic acid, octynoic acid, a saturated or unsaturated alicyclic carboxylic acid, cyclobutanecarboxylic acid, cyclopentanecarboxylic acid, cyclopentenecarboxylic acid, methylcyclopentenecarboxylic acid, cyclohexanecarboxylic acid, dimethylcyclohexanecarboxylic acid, dipropylcyclohexanecarboxylic acid, a saturated or unsaturated, alicyclic aliphatic carboxylic acid, cyclopentaneacetic acid, cyclopentanepropionic acid, cyclohexaneacetic acid, cyclohexanebutyric acid, methylcyclohexaneacetic acid, a substituted or unsubstituted aromatic carboxylic acid, benzoic acid, toluic acid, naphthoic acid, ethylbenzoic acid, isobutylbenzoic acid, methylbutylbenzoic acid, an aromatic aliphatic carboxylic acid, phenylacetic acid, phenylpropionic acid, phenylvaleric acid, cinnamic acid, phenylpropiolic acid, naphthylacetic acid, a halo-alkoxyhydrocarbon carboxylic acid, a nitro-alkoxyhydrocarbon carboxylic acid, a hydroxy-alkoxyhydrocarbon carboxylic acid, an amino-alkoxyhydrocarbon carboxylic acid, a cyano-alkoxyhydrocarbon carboxylic acid, a thiocyano-alkoxyhydrocarbon carboxylic acid, mono-acetic acid, di-acetic acid, trichloroacetic acid, 1,2,3,4,5,6-hexachlorocyclohexanecarboxylic acid, 1,2-dibromo-4-methylcyclohexanecarboxylic acid, 1,6-dibromo-3-methylcyclohexanecarboxylic acid, 1-bromo-3,5-dimethylcyclohexanecarboxylic acid, 2-chlorocyclohexanecarboxylic acid, 4-chlorocyclohexanecarboxylic acid, 2,3-dibromo-2-methylcyclohexanecarboxylic acid, 2,4,6-trinitrobenzoic acid, 2,5-dibromo-2-methylcyclohexanecarboxylic acid, 2-bromo-4-methylcyclohexanecarboxylic acid, 2-nitro-1-methyl-cyclobutanecarboxylic acid, 3,4-dinitrobenzoic acid, 3,5-dinitrobenzoic acid, 3-bromo-2,2,3-trimethylcyclopentanecarboxylic acid, 3-bromo-2-methylcyclohexanecarboxylic acid, 3-bromo-3-methylcyclohexanecarboxylic acid, 4-bromo-2-methylcyclohexanecarboxylic acid, 5-bromo-2-methylcyclohexanecarboxylic acid, ‘4,4-dichlorobenzilic acid, 4,5-dibromo-2-methylcyclohexanecarboxylic acid, 5-bromo-2-methylcyclohexanecarboxylic acid, 6-bromo-2-methylcyclohexanecarboxylic acid, 5,6-dibromo-2-methylcyclohexanecarboxylic acid, 6-bromo-3-methylcyclohexanecarboxylic acid, anisic acid, cyanoacetic acid, cyanopropionic acid, ethoxyformic acid (ethyl hydrogen carbonate), gallic acid, homogentisic acid, o-, m-, and p-chlorobenzoic acid, lactic acid, mevalonic acid, o-, m-, p-nitrobenzoic acid, p-hydroxybenzoic acid, salicyclic acid, shikimic acid, thiocyanoacetic acid, trimethoxybenzoic acid, trimethoxycinnamic acid, veratric acid, α- and β-chloropropionic acid, α- and γ-bromobutyric acid and α- and δ-iodovaleric acid, β-resorcylic acid.

79. A pharmaceutical composition comprising the compound of claim 50 and a pharmaceutically acceptable carrier.

80. A method of treating a medical condition associated with a pathogenic microorganism in a subject in need thereof, the method comprising administering to the subject a therapeutically effective amount of the compound of claim 50.
